SEQUENCE LISTING (156 sequences)

SEQ ID NO: 1 - protein, 118 aa, Artificial Sequence
  45F1F3 Variable Heavy Chain Sequence
DVQLQESGPGLVKPSQSLSLTCTVTGYSITIDYAWNWIRQFPGNKLEWMGYISNSGYTSYNPSLKSRISFTRDTSKNQFFLQLNSVTPEDSATYYCTRSGHYDLFDYWGQGTTLTVSS

SEQ ID NO: 2 - protein, 112 aa, Artificial Sequence
  45F1F3 Variable Light Chain Sequence
DVVMTQAPLTLSVTIGQPASISCKSSQSLFDRDGKTYLSWLLERPGQSPKRLIYLVSNLDSGVPDRFTGSGSGTDFTLKISRVEAEDLGVYYCWQGTHFPYTFGGGTKVEMK

SEQ ID NO: 3 - protein, 118 aa, Artificial Sequence
  225H7D12 Variable Heavy Chain Sequence
QVQLQQSGAEVMKPGASVKLSCKTTGYRFTGYWIEWVKQRPGHGLEWIGEILPGSGVSNNNEKFRDKATFTADTSSNTAYIQLSSLTTDDSAIYYCARKASYYTMDYWGQGTSVTVSS

SEQ ID NO: 4 - protein, 108 aa, Artificial Sequence
  225H7D12 Variable Light Chain Sequence
QIVLTQSPAIMSASLGERVTMTCTASSSVSSFYFHWYQQKPGSSPKLWIYSISNLASGVPTRFSGSGSGTSYSLTISSMEAEDAATYYCHQYHRSPRTFGGGTKLEIK

SEQ ID NO: 5 - protein, 117 aa, Artificial Sequence
  270C5C10 Ha Variable Heavy Chain Sequence
EVQLQQSGPEVVKPGASVKISCKASGYTFTDNYMNWAKQSQGKSLEWIGDINPNNGGSRYKEKFKDKATLTVDKSSRTAYMELRSLTSEDSAVYYCANPGSYGFAYWGQGTLVTVSA

SEQ ID NO: 6 - protein, 117 aa, Artificial Sequence
  270C5C10 Hb Variable Heavy Chain Sequence
EVQLQQSGPVQVKPGASVKMSCKASGFTFTDYYMNWVKQSHGKSLEWIGIINPNNGDTSYNQKFKGKATLTVDKSSSTAYMELNSLTSEDSAVYYCARRDYDWFAYWGQGTLVTVSA

SEQ ID NO: 7 - protein, 112 aa, Artificial Sequence
  270C5C10 Variable Light Chain Sequence
DVVLTQTPLTLSVTLGQPASISCKSSQSLLDSDGRTYLNWLLQRPGQSPKRLIYLVSQLDSGVPDRFTGSGSGTDFTLKISRVEAEDLGVYFCWQGTHFPWTFGGGTKLEIK

SEQ ID NO: 8 - protein, 121 aa, Artificial Sequence
  134D7B3 Variable Heavy Chain Sequence
QVQLQQSGAELVRPGTSVKVSCKASGYAFNNYVIEWVKQRPGQGLEWIGVIYPGNGGSDYNEKFKVKAKLTADKSSSTAYMQLSSLTSDDSAVYFCARFYYFGSSYAMDYWGQGTSVTVSS

SEQ ID NO: 9 - protein, 107 aa, Artificial Sequence
  134D7B3 Variable Light Chain Sequence
DIQMTQTTSSLSASLGDRVTISCRASQDISNYLNWYQQKPDGTIKLLIYYTSRLHSGVPSRFSGSGSGTDYSLTITNLEQEDIATYFCQQGNTLPFTFGSGTKLEIK

SEQ ID NO: 10 - protein, 123 aa, Artificial Sequence
  177C6B11 Variable Heavy Chain Sequence
QVQLQQPGAELVKPGASVKLSCKASGYTFTSFWMHWVKQRPGRGLEWIGRIDPHTGGTKYNEKFKTKATLTVDKPSSAAYMQLSSLTSEDSAVYYCARGGYYDYDGVAWLAFWGQGTLVTVSA

SEQ ID NO: 11 - protein, 113 aa, Artificial Sequence
  177C6B11 Variable Light Chain Sequence
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEIK

SEQ ID NO: 12 - protein, 115 aa, Artificial Sequence
  215F2A5 Variable Heavy Chain Sequence
EVQLQQSGPDLVKPGTSVKISCKASGYTLSDHYMTWVKQSHGKSLEWIADINPYSGTVTFNQRFKDKATLTVDTSSSTAFMELRSLTSEDSAVYYCARREAMDYWGQGTSVTVSS

SEQ ID NO: 13 - protein, 112 aa, Artificial Sequence
  215F2A5 Variable Light Chain Sequence
GVLMTQTPLSLPVSLGDQASISCRSDQTIVYSNGNTYLQWYLQRPGQSPRLLIYKVSNRFSGVPDRFSGSGSGTDFTLKISRVEAEDLGVYYCFQGSHVPYTFGGGTRLEIK

SEQ ID NO: 14 - protein, 115 aa, Artificial Sequence
  223H11H1 Variable Heavy Chain Sequence
EVQLQQSGPELVKPGASVKISCKASGYTFTAHYMNWVKKSQGKSLEWIGDINPDNGIIRYNQKFKDKATLTVDKSSSTVFLEFRSLASEDSAVYYCARREALDYWGQGSSVTVSS

SEQ ID NO: 15 - protein, 112 aa, Artificial Sequence
  223H11H1 Variable Light Chain Sequence
DVLMTQIPLSLTVSLGDQASISCRSSQSIVYSNGKVYLNWYQQKPGQSPKLLIYKVSDRFSGVPDRFSGSGSGTDFTLKINRVEAEDLGVYYCLQGSHIPWTFGGGTKLEIK

SEQ ID NO: 16 - protein, 118 aa, Artificial Sequence
  204C10G12 Variable Heavy Chain Sequence
QVQLQQPGAELVKPGASVKMSCKASGYTFTSYWITWVKQRPGQGLEWIGDIYPGSDSTNYNEKFKNKATLTVDTSSSTAYMQLSSLTSEDSAVYYCARGDDFWYFDVWGTGTTVTVSS

SEQ ID NO: 17 - protein, 112 aa, Artificial Sequence
  204C10G12 Variable Light Chain Sequence
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 18 - protein, 117 aa, Artificial Sequence
  206H9G11 Ha Variable Heavy Chain Sequence
EVQLQQSGPALVKPGASVKISCKASGYSFTDYYMHWMKQSPEKSLEWIGEINPSTGGTSYNQKFKAKATLTVDKSSNTAYMQLKSLTSEDSAVYFCTRRDYYAMDYWDQGTSVIVSS

SEQ ID NO: 19 - protein, 125 aa, Artificial Sequence
  206H9G11 Hb Variable Heavy Chain Sequence
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIAPITTVVATNYFDVWGTGTTVTVSS

SEQ ID NO: 20 - protein, 107 aa, Artificial Sequence
  206H9G11 La Variable Light Chain Sequence
DIVMTQSHKFMSTSVGDRVNITCKASQDVRIAVAWYQQKPGQSPKLLIYSASYRFTGVPDRFTGSGSGTDFTFTISSVQAEDLAVYYCQQHYSFPLTFGAGTKLELT

SEQ ID NO: 21 - protein, 106 aa, Artificial Sequence
  206H9G11 Lb Variable Light Chain Sequence
DILLTQSPAILSVSPGERVSFSCRASQSIGTSIHWYQQRTNGSPRLLIKYASESISGIPSRFSGSGSGTDFTLSINSVESEDIADYYCQQSNSWLTFGAGTKLELK

SEQ ID NO: 22 - protein, 121 aa, Artificial Sequence
  225A8D9 Variable Heavy Chain Sequence
QVQLQQSGPELVKPGASVKISCKASGYAFSSSWMNWVKQRPGKGLEWIGRIYPGDGNINYDGKFKGEATLTADKSSSTAYIQLSSLTSEDSAVYFCARSAWLGKTYVMDYWGQGTSVTVSS

SEQ ID NO: 23 - protein, 105 aa, Artificial Sequence
  225A8D9 Variable Light Chain Sequence
EIVVTQSPAITAASLGQKVTITCSASSSVTYMNWYQQKSGTSPKPWIYEISKLASGVPAHFSGSGSGTSYSLTISSLEAEDAAIYYCQQWNFPFTFGSGTKVEIR

SEQ ID NO: 24 - protein, 120 aa, Artificial Sequence
  274C7H2 Variable Heavy Chain Sequence
QVQLQQSGAELAKPGASVKLSCKASGYTFTSYWMHWVKQRPGQGLEWIGYINPSSGYTKYNQKFKDKATLTADKSSSTAYMQLSSLTYEDSAVYYCARNPSYDYYAMDYWGQGTSVTVSS

SEQ ID NO: 25 - protein, 109 aa, Artificial Sequence
  274C7H2 Variable Light Chain Sequence
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHFIFGSGTKVTVL

SEQ ID NO: 26 - protein, 116 aa, Artificial Sequence
  384H3H11 Variable Heavy Chain Sequence
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARIGLRSFAYWGQGTLVTVSA

SEQ ID NO: 27 - protein, 113 aa, Artificial Sequence
  384H3H11 Variable Light Chain Sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 28 - protein, 117 aa, Artificial Sequence
  163H12G7 Variable Heavy Chain Sequence
QVQLQQSGAELASPGASVKMSCKASGYTFSTYTVHWVKQRPGQGLEWIGYINPASGYTNYNQKFKDRATLTADRSSTTAYMQLSGLTSEDSAVFYCARWSYWYFDVWGTGTTVTVSS

SEQ ID NO: 29 - protein, 112 aa, Artificial Sequence
  163H12G7 Variable Light Chain Sequence
DVVMTQTPLTLSVSIGQPASISCKSSQSLLDSDGKTYLNWFLQRPGQSPKRLLYLVSKLDSGVPDRFTGSGSGTDFTLKISRVEAEDLGVYYCYQATHFPLTFGAGTKLELK

SEQ ID NO: 30 - protein, 124 aa, Artificial Sequence
  172H7B9 Variable Heavy Chain Sequence
QVTLKESGPGILQSSQTLSLTCSFSGFSLSPSGMGVTWIRQPSGKGLEWLAHIYWDDVKRYNPSLKSRLTISKDTSRNQVFLKITGVDTADTATYYCARRGSYSDYGGWSFDVWGTGTTVTVSS

SEQ ID NO: 31 - protein, 113 aa, Artificial Sequence
  172H7B9 La Variable Light Chain Sequence
DIVMTQSPSSLSVSAGEKVTMSCKSSQSLFNSGNQKNYLAWYQQKPGQPPKLLIYGASTRESGVPDRFTGSGSGSDFTLTISSVQAEDLAVYYCQNDHSYPPTFGSGTKLEIK

SEQ ID NO: 32 - protein, 113 aa, Artificial Sequence
  172H7B9 Lb Variable Light Chain Sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 33 - protein, 113 aa, Artificial Sequence
  172H7B9 Lc Variable Light Chain Sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDHSYPPTFGSGTKLEIK

SEQ ID NO: 34 - protein, 114 aa, Artificial Sequence
  223F12C4 Variable Heavy Chain Sequence
QVQLQQSGAELVKPGASVKVSCKASGYSFTSYWIHWVKQRPGQGLEWIGRIHPSDGDIDHNEKFKGKATLTVDKSSSTAYMQLTSLTSEDSAVYYCLCYIMDYWGQGTSVTVSS

SEQ ID NO: 35 - protein, 107 aa, Artificial Sequence
  223F12C4 Variable Light Chain Sequence
DIQMNQSPSSLSASLGDTIAITCHASQNINVWLSWYQQKPGNIPKLLIYKAAKLQTGVPSRFSGSGSGTGFTLTISSLQPEDIATYYCHQGQDYPLTFGAGTKLELK

SEQ ID NO: 36 - protein, 6 aa, Artificial Sequence
  45F1F3 HCDR1
IDYAWN

SEQ ID NO: 37 - protein, 16 aa, Artificial Sequence
  45F1F3 HCDR2
YISNSGYTSYNPSLKS

SEQ ID NO: 38 - protein, 9 aa, Artificial Sequence
  45F1F3 HCDR3
SGHYDLFDY

SEQ ID NO: 39 - protein, 16 aa, Artificial Sequence
  45F1F3 LCDR1
KSSQSLFDRDGKTYLS

SEQ ID NO: 40 - protein, 7 aa, Artificial Sequence
  45F1F3 LCDR2
LVSNLDS

SEQ ID NO: 41 - protein, 9 aa, Artificial Sequence
  45F1F3 LCDR3
WQGTHFPYT

SEQ ID NO: 42 - protein, 5 aa, Artificial Sequence
  225H7D12 HCDR1
GYWIE

SEQ ID NO: 43 - protein, 17 aa, Artificial Sequence
  225H7D12 HCDR2
EILPGSGVSNNNEKFRD

SEQ ID NO: 44 - protein, 9 aa, Artificial Sequence
  225H7D12 HCDR3
KASYYTMDY

SEQ ID NO: 45 - protein, 12 aa, Artificial Sequence
  225H7D12 LCDR1
TASSSVSSFYFH

SEQ ID NO: 46 - protein, 7 aa, Artificial Sequence
  225H7D12 LCDR2
SISNLAS

SEQ ID NO: 47 - protein, 9 aa, Artificial Sequence
  225H7D12 LCDR3
HQYHRSPRT

SEQ ID NO: 48 - protein, 5 aa, Artificial Sequence
  270C5C10 Ha HCDR1
DNYMN

SEQ ID NO: 49 - protein, 17 aa, Artificial Sequence
  270C5C10 Ha HCDR2
DINPNNGGSRYKEKFKD

SEQ ID NO: 50 - protein, 8 aa, Artificial Sequence
  270C5C10 Ha HCDR3
PGSYGFAY

SEQ ID NO: 51 - protein, 5 aa, Artificial Sequence
  270C5C10 Hb HCDR1
DYYMN

SEQ ID NO: 52 - protein, 17 aa, Artificial Sequence
  270C5C10 Hb HCDR2
IINPNNGDTSYNQKFKG

SEQ ID NO: 53 - protein, 8 aa, Artificial Sequence
  270C5C10 Hb HCDR3
RDYDWFAY

SEQ ID NO: 54 - protein, 16 aa, Artificial Sequence
  270C5C10 LCDR1
KSSQSLLDSDGRTYLN

SEQ ID NO: 55 - protein, 7 aa, Artificial Sequence
  270C5C10 LCDR2
LVSQLDS

SEQ ID NO: 56 - protein, 9 aa, Artificial Sequence
  270C5C10 LCDR3
WQGTHFPWT

SEQ ID NO: 57 - protein, 5 aa, Artificial Sequence
  134D7B3 HCDR1
NYVIE

SEQ ID NO: 58 - protein, 17 aa, Artificial Sequence
  134D7B3 HCDR2
VIYPGNGGSDYNEKFKV

SEQ ID NO: 59 - protein, 12 aa, Artificial Sequence
  134D7B3 HCDR3
FYYFGSSYAMDY

SEQ ID NO: 60 - protein, 11 aa, Artificial Sequence
  134D7B3 LCDR1
RASQDISNYLN

SEQ ID NO: 61 - protein, 7 aa, Artificial Sequence
  134D7B3 LCDR2
YTSRLHS

SEQ ID NO: 62 - protein, 9 aa, Artificial Sequence
  134D7B3 LCDR3
QQGNTLPFT

SEQ ID NO: 63 - protein, 5 aa, Artificial Sequence
  177C6B11 HCDR1
SFWMH

SEQ ID NO: 64 - protein, 17 aa, Artificial Sequence
  177C6B11 HCDR2
RIDPHTGGTKYNEKFKT

SEQ ID NO: 65 - protein, 14 aa, Artificial Sequence
  177C6B11 HCDR3
GGYYDYDGVAWLAF

SEQ ID NO: 66 - protein, 17 aa, Artificial Sequence
  177C6B11 LCDR1
KSSQSLLYSSNQKNYLA

SEQ ID NO: 67 - protein, 7 aa, Artificial Sequence
  177C6B11 LCDR2
WASTRES

SEQ ID NO: 68 - protein, 9 aa, Artificial Sequence
  177C6B11 LCDR3
QQYYSYPWT

SEQ ID NO: 69 - protein, 5 aa, Artificial Sequence
  215F2A5 HCDR1
DHYMT

SEQ ID NO: 70 - protein, 17 aa, Artificial Sequence
  215F2A5 HCDR2
DINPYSGTVTFNQRFKD

SEQ ID NO: 71 - protein, 6 aa, Artificial Sequence
  215F2A5 HCDR3
REAMDY

SEQ ID NO: 72 - protein, 16 aa, Artificial Sequence
  215F2A5 LCDR1
RSDQTIVYSNGNTYLQ

SEQ ID NO: 73 - protein, 7 aa, Artificial Sequence
  215F2A5 LCDR2
KVSNRFS

SEQ ID NO: 74 - protein, 9 aa, Artificial Sequence
  215F2A5 LCDR3
FQGSHVPYT

SEQ ID NO: 75 - protein, 5 aa, Artificial Sequence
  223H11H1 HCDR1
AHYMN

SEQ ID NO: 76 - protein, 17 aa, Artificial Sequence
  223H11H1 HCDR2
DINPDNGIIRYNQKFKD

SEQ ID NO: 77 - protein, 6 aa, Artificial Sequence
  223H11H1 HCDR3
REALDY

SEQ ID NO: 78 - protein, 16 aa, Artificial Sequence
  223H11H1 LCDR1
RSSQSIVYSNGKVYLN

SEQ ID NO: 79 - protein, 7 aa, Artificial Sequence
  223H11H1 LCDR2
KVSDRFS

SEQ ID NO: 80 - protein, 9 aa, Artificial Sequence
  223H11H1 LCDR3
LQGSHIPWT

SEQ ID NO: 81 - protein, 5 aa, Artificial Sequence
  204C10G12 HCDR1
SYWIT

SEQ ID NO: 82 - protein, 17 aa, Artificial Sequence
  204C10G12 HCDR2
DIYPGSDSTNYNEKFKN

SEQ ID NO: 83 - protein, 9 aa, Artificial Sequence
  204C10G12 HCDR3
GDDFWYFDV

SEQ ID NO: 84 - protein, 16 aa, Artificial Sequence
  204C10G12 LCDR1
RSSQSIVHSNGNTYLE

SEQ ID NO: 85 - protein, 7 aa, Artificial Sequence
  204C10G12 LCDR2
KVSNRFS

SEQ ID NO: 86 - protein, 9 aa, Artificial Sequence
  204C10G12 LCDR3
FQGSHVPLT

SEQ ID NO: 87 - protein, 5 aa, Artificial Sequence
  206H9G11 H1 HCDR1
DYYMH

SEQ ID NO: 88 - protein, 17 aa, Artificial Sequence
  206H9G11 H1 HCDR2
EINPSTGGTSYNQKFKA

SEQ ID NO: 89 - protein, 8 aa, Artificial Sequence
  206H9G11 H1 HCDR3
RDYYAMDY

SEQ ID NO: 90 - protein, 7 aa, Artificial Sequence
  206H9G11 H2 HCDR1
TFGMGVG

SEQ ID NO: 91 - protein, 16 aa, Artificial Sequence
  206H9G11 H2 HCDR2
HIWWDDDKYYNPALKS

SEQ ID NO: 92 - protein, 15 aa, Artificial Sequence
  206H9G11 H2 HCDR3
IAPITTVVATNYFDV

SEQ ID NO: 93 - protein, 11 aa, Artificial Sequence
  206H9G11 L1 LCDR1
KASQDVRIAVA

SEQ ID NO: 94 - protein, 7 aa, Artificial Sequence
  206H9G11 L1 LCDR2
SASYRFT

SEQ ID NO: 95 - protein, 9 aa, Artificial Sequence
  206H9G11 L1 LCDR3
QQHYSFPLT

SEQ ID NO: 96 - protein, 11 aa, Artificial Sequence
  206H9G11 L2 LCDR1
RASQSIGTSIH

SEQ ID NO: 97 - protein, 7 aa, Artificial Sequence
  206H9G11 L2 LCDR2
YASESIS

SEQ ID NO: 98 - protein, 8 aa, Artificial Sequence
  206H9G11 L2 LCDR3
QQSNSWLT

SEQ ID NO: 99 - protein, 5 aa, Artificial Sequence
  225A8D9 HCDR1
SSWMN

SEQ ID NO: 100 - protein, 17 aa, Artificial Sequence
  225A8D9 HCDR2
RIYPGDGNINYDGKFKG

SEQ ID NO: 101 - protein, 12 aa, Artificial Sequence
  225A8D9 HCDR3
SAWLGKTYVMDY

SEQ ID NO: 102 - protein, 10 aa, Artificial Sequence
  225A8D9 LCDR1
SASSSVTYMN

SEQ ID NO: 103 - protein, 7 aa, Artificial Sequence
  225A8D9 LCDR2
EISKLAS

SEQ ID NO: 104 - protein, 8 aa, Artificial Sequence
  225A8D9 LCDR3
QQWNFPFT

SEQ ID NO: 105 - protein, 5 aa, Artificial Sequence
  274C7H2 HCDR1
SYWMH

SEQ ID NO: 106 - protein, 17 aa, Artificial Sequence
  274C7H2 HCDR2
YINPSSGYTKYNQKFKD

SEQ ID NO: 107 - protein, 11 aa, Artificial Sequence
  274C7H2 HCDR3
NPSYDYYAMDY

SEQ ID NO: 108 - protein, 14 aa, Artificial Sequence
  274C7H2 LCDR1
RSSTGAVTTSNYAN

SEQ ID NO: 109 - protein, 7 aa, Artificial Sequence
  274C7H2 LCDR2
GTNNRAP

SEQ ID NO: 110 - protein, 9 aa, Artificial Sequence
  274C7H2 LCDR3
ALWYSNHFI

SEQ ID NO: 111 - protein, 5 aa, Artificial Sequence
  383H3H11 HCDR1
SYGVH

SEQ ID NO: 112 - protein, 16 aa, Artificial Sequence
  383H3H11 HCDR2
VIWSGGSTDYNAAFIS

SEQ ID NO: 113 - protein, 8 aa, Artificial Sequence
  383H3H11 HCDR3
IGLRSFAY

SEQ ID NO: 114 - protein, 17 aa, Artificial Sequence
  383H3H11 LCDR1
KSSQSLLNSGNQKNYLT

SEQ ID NO: 115 - protein, 7 aa, Artificial Sequence
  383H3H11 LCDR2
WASTRES

SEQ ID NO: 116 - protein, 9 aa, Artificial Sequence
  383H3H11 LCDR3
QNDYSYPFT

SEQ ID NO: 117 - protein, 5 aa, Artificial Sequence
  163H12G7 HCDR1
TYTVH

SEQ ID NO: 118 - protein, 17 aa, Artificial Sequence
  163H12G7 HCDR2
YINPASGYTNYNQKFKD

SEQ ID NO: 119 - protein, 8 aa, Artificial Sequence
  163H12G7 HCDR3
WSYWYFDV

SEQ ID NO: 120 - protein, 16 aa, Artificial Sequence
  163H12G7 LCDR1
KSSQSLLDSDGKTYLN

SEQ ID NO: 121 - protein, 7 aa, Artificial Sequence
  163H12G7 LCDR2
LVSKLDS

SEQ ID NO: 122 - protein, 9 aa, Artificial Sequence
  163H12G7 LCDR3
YQATHFPLT

SEQ ID NO: 123 - protein, 7 aa, Artificial Sequence
  172H7B9 HCDR1
PSGMGVT

SEQ ID NO: 124 - protein, 16 aa, Artificial Sequence
  172H7B9 HCDR2
HIYWDDVKRYNPSLKS

SEQ ID NO: 125 - protein, 14 aa, Artificial Sequence
  172H7B9 HCDR3
RGSYSDYGGWSFDV

SEQ ID NO: 126 - protein, 17 aa, Artificial Sequence
  172H7B9 La LCDR1
KSSQSLFNSGNQKNYLA

SEQ ID NO: 127 - protein, 7 aa, Artificial Sequence
  172H7B9 La LCDR2
GASTRES

SEQ ID NO: 128 - protein, 9 aa, Artificial Sequence
  172H7B9 La LCDR3
QNDHSYPPT

SEQ ID NO: 129 - protein, 17 aa, Artificial Sequence
  172H7B9 Lb LCDR1
KSSQSLLNSGNQKNYLT

SEQ ID NO: 130 - protein, 7 aa, Artificial Sequence
  172H7B9 Lb LCDR2
WASTRES

SEQ ID NO: 131 - protein, 9 aa, Artificial Sequence
  172H7B9 Lb LCDR3
QNDYSYPLT

SEQ ID NO: 132 - protein, 17 aa, Artificial Sequence
  172H7B9 Lc LCDR1
KSSQSLLNSGNQKNYLT

SEQ ID NO: 133 - protein, 7 aa, Artificial Sequence
  172H7B9 Lc LCDR2
WASTRES

SEQ ID NO: 134 - protein, 9 aa, Artificial Sequence
  172H7B9 Lc LCDR3
QNDHSYPPT

SEQ ID NO: 135 - protein, 5 aa, Artificial Sequence
  223F12C4 HCDR1
SYWIH

SEQ ID NO: 136 - protein, 17 aa, Artificial Sequence
  223F12C4 HCDR2
RIHPSDGDIDHNEKFKG

SEQ ID NO: 137 - protein, 5 aa, Artificial Sequence
  223F12C4 HCDR3
YIMDY

SEQ ID NO: 138 - protein, 11 aa, Artificial Sequence
  223F12C4 LCDR1
HASQNINVWLS

SEQ ID NO: 139 - protein, 7 aa, Artificial Sequence
  223F12C4 LCDR2
KAAKLQT

SEQ ID NO: 140 - protein, 9 aa, Artificial Sequence
  223F12C4 LCDR3
HQGQDYPLT

SEQ ID NO: 141 - protein, 330 aa, Artificial Sequence [sequence omitted]

SEQ ID NO: 142 - protein, 107 aa, Artificial Sequence [sequence omitted]

SEQ ID NO: 143 - protein, 118 aa, Artificial Sequence
  45F1F3 VH1.1
QVQLQESGPGLVKPSQTLSLTCTVTGYSITIDYAWNWIRQPPGKGLEWIGYISNSGYTSYNPSLKSRVTFSRDTSKNQFSLKLSSVTAADTAVYYCTRSGHYDLFDYWGQGTTVTVSS

SEQ ID NO: 144 - protein, 118 aa, Artificial Sequence
  45F1F3 VH1.3
QVQLQESGPGLVKPSQTLSLTCTVTGYSITIDYAWNWIRQPPGKGLEWMGYISNSGYTSYNPSLKSRVTISRDTSKNQFFLKLSSVTAADTAVYYCTRSGHYDLFDYWGQGTTVTVSS

SEQ ID NO: 145 - protein, 118 aa, Artificial Sequence
  45F1F3 VH1.4
DVQLQESGPGLVKPSQTLSLTCTVTGYSITIDYAWNWIRQPPGKGLEWMGYISNSGYTSYNPSLKSRITFSRDTSKNQFFLKLSSVTAADTAVYYCTRSGHYDLFDYWGQGTTVTVSS

SEQ ID NO: 146 - protein, 112 aa, Artificial Sequence
  45F1F3 L1
DVVMTQSPLSLPVTLGQPASISCKSSQSLFDRDGKTYLSWFQQRPGQSPRRLIYLVSNLDSGVPDRFSGSGSGTDFTLKISRVEAEDVGVYYCWQGTHFPYTFGGGTKVEIK

SEQ ID NO: 147 - protein, 112 aa, Artificial Sequence
  45F1F3 L1.1
DVVMTQSPLSLPVTLGQPASISCKSSQSLFDRDGKTYLSWLLQRPGQSPRRLIYLVSNLDSGVPDRFSGSGSGTDFTLKISRVEAEDVGVYYCWQGTHFPYTFGGGTKVEIK

SEQ ID NO: 148 - protein, 118 aa, Artificial Sequence
  225H7D12 VH1.1
QVQLVQSGAEVKKPGASVKVSCKTTGYRFTGYWIEWVRQAPGQGLEWIGEILPGSGVSNNNEKFRDRATMTADTSISTAYMELSRLRSDDTAVYYCARKASYYTMDYWGQGTLVTVSS

SEQ ID NO: 149 - protein, 118 aa, Artificial Sequence
  225H7D12 VH1.3
QVQLVQSGAEVKKPGASVKVSCKASGYRFTGYWIEWVKQAPGQGLEWIGEILPGSGVSNNNEKFRDRATMTADTSINTAYMELSRLRSDDTAVYYCARKASYYTMDYWGQGTLVTVSS

SEQ ID NO: 150 - protein, 118 aa, Artificial Sequence
  225H7D12 VH1.4
QVQLVQSGAEVKKPGASVKVSCKTTGYRFTGYWIEWVKQAPGQGLEWIGEILPGSGVSNNNEKFRDRATFTADTSINTAYMELSRLRSDDTAVYYCARKASYYTMDYWGQGTLVTVSS

SEQ ID NO: 151 - protein, 108 aa, Artificial Sequence
  225H7D12 L1.2
DIQMTQSPSSLSASVGDRVTITCTASSSVSSFYFHWYQQKPGKAPKLWIYSISNLASGVPSRFSGSGSGTSYTLTISSLQPEDFATYYCHQYHRSPRTFGGGTKLEIK

SEQ ID NO: 152 - protein, 117 aa, Artificial Sequence
  270C5C10 VHa1.2
QVQLVQSGAEVKKPGASVKVSCKASGYTFTDNYMNWVRQAPGQSLEWIGDINPNNGGSRYKEKFKDRATLTVDTSIRTAYMELSRLRSDDTAVYYCANPGSYGFAYWGQGTLVTVSS

SEQ ID NO: 153 - protein, 117 aa, Artificial Sequence
  270C5C10 VHa1.4
QVQLVQSGAEVKKPGASVKVSCKASGYTFTDNYMNWARQSPGQSLEWIGDINPNNGGSRYKEKFKDRATLTVDTSIRTAYMELSRLRSDDTAVYYCANPGSYGFAYWGQGTLVTVSS

SEQ ID NO: 154 - protein, 112 aa, Artificial Sequence
  270C5C10 VL1
DVVMTQSPLSLPVTLGQPASISCKSSQSLLDSDGRTYLNWFQQRPGQSPRRLIYLVSQLDSGVPDRFSGSGSGTDFTLKISRVEAEDVGVYYCWQGTHFPWTFGGGTKLEIK

SEQ ID NO: 155 - protein, 329 aa, Artificial Sequence
  Human IgG1 CH variant 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 330 aa, Artificial Sequence
  Human IgG1 CH variant 2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0064703 A1
(43) Pub. Date: Mar. 2, 2023

(54) ANTI-GITR ANTIBODIES AND USES THEREOF

(71) Applicant: NANJING GENSCRIPT BIOTECH CO., LTD., Nanjing (CN)

(72) Inventors: Liusong Yin, Nanjing (CN); Zhuo Fang, Nanjing (CN); Wenshuang Jia, Nanjing (CN); Zhaoyang Wei, Nanjing (CN)

(73) Assignee: NANJING GENSCRIPT BIOTECH CO., LTD., Nanjing (CN)

(21) Appl. No.: 17/790,937

(22) PCT Filed: Dec. 31, 2020
(86) PCT No.: PCT/CN2020/142113

§ 371 Date: Jul. 5, 2022

(30) Foreign Application Priority Data
Jan. 2, 2020 (CN) PCT/CN2020/070101

(51) Int. Cl. C07K 16/28; C12N 15/63; A61P 35/00

(52) U.S. Cl. CPC C07K 16/2878; C12N 15/63; A61P 35/00; C07K 2317/565; C07K 2317/24

(57) ABSTRACT

Anti-GITR antibodies and antigen-binding fragments thereof are described. Also described are nucleic acids encoding the antibodies, compositions comprising the antibodies, methods of producing the antibodies, and methods of using the antibodies for treating or preventing diseases, such as cancer.

### FIELD OF THE INVENTION

This invention relates to antibodies or antigen binding fragments thereof capable of binding specifically to a GITR protein, preferably a human GITR protein, and uses of such agents. In some embodiments, the application relates to mouse and humanized monoclonal antibodies directed to GITR and uses of these antibodies. The antibodies or antigen binding fragments thereof are useful as diagnostics and for the treatment of diseases associated with the activity and/or expression of GITR.

### REFERENCE TO SEQUENCE LISTING SUBMITTED ELECTRONICALLY

This application contains a sequence listing, which is submitted electronically via EFS-Web as an ASCII formatted sequence listing with a file name “688096.129 Sequence Listing” and a creation date of Dec. 28, 2019 and having a size of 82 kb. The sequence listing submitted via EFS-Web is part of the specification and is herein incorporated by reference in its entirety.

### BACKGROUND OF THE INVENTION

Cancer immunotherapy harnesses the body's own immune system by stimulating, amplifying, or supplementing the immune system to better recognize, manage, and even reverse the disease phenotype. One of the most exploited classes of immunotherapy involves targeting immune checkpoints, such as program cell death protein 1 (PD-1), cytotoxic T-lymphocyte-associated protein 4 (CTLA4), T-cell immunoglobulin and mucin-domain containing-3 (TIM-3), lymphocyte-activation gene 3 (LAG-3), and V-domain Ig suppressor of T cell activation (VISTA), the key regulators that dampen the immune response. The discovery of antibodies as immune checkpoint inhibitors has been one of the most successful approach in cancer drug discovery. Following the approval of ipilimumab in 2011, the first checkpoint inhibitor targeting CTLA4, immune checkpoint inhibitors are now being offered as a frontline treatment for multiple cancers, such as metastatic melanoma, non-small cell lung cancer (NSCLC), renal cell carcinoma (RCCs), breast cancer, urothelial carcinoma, and liver cancer.

Despite the revolutionary success of immune checkpoint blockade, there is still a proportion of patients that do not benefit or show any positive response. As a result, developing alternative therapeutic options is still a major focus to further enhance antitumor immunity. Among which, glucocorticoid-induced tumor necrosis factor receptor related protein (GITR) has gathered substantial interest as an attractive target for immunotherapy.

GITR belongs to the tumor necrosis factor receptor superfamily (TNFRSF), which are cytokine receptors that bind to tumor necrosis factors (TNFs) via an extracellular cysteine-rich domain. TNFRSF are involved in diverse cellular processes from apoptosis and inflammation to other signal transduction pathways in proliferation, survival, and differentiation. GITR was identified as a member of the TNFRSF, which protects T cells from apoptosis upon the exposure to glucocorticoid hormone (GC), a potent inducer of the death of T cells and commonly used as an immunosuppressive and anti-inflammatory agent. GITR is constitutively and exclusively expressed at high levels on CD25⁺CD4⁺ regulatory T cells, its ligand, GITRL is predominantly expressed by activated antigen presenting cells (APCs). The expression of GITR and GITRL has also been noted on epidermal keratinocytes, osteoclast precursors, and epithelial cells. This suggests that the function of GITR and GITRL may not be limited to regulating immune responses but also involve mediating leukocyte adhesion and migration. The anticancer therapeutic potential in modulating GITR function is attributed to its costimulatory role in effector T cells (T_{eff}) and the inhibition or depletion of tumor infiltrating regulatory T (T_{reg}) cells. Preclinical tumor models using an agonist anti-mouse GITR monoclonal antibody, namely DTA-1, have elucidated its effects, which include increased infiltration of CD4⁺, CD8⁺ T, and CD4⁺IL9⁺ (Th9) cells; decreased levels of CD4⁺FoxP3⁺ T_{regs}; and the overall enhancement of the T_{eff} to T_{reg} ratio.

Preclinical combination of anti-GITR with other treatment modalities such as anti-PD-1 and anti-CTLA4 have also demonstrated promising results by potentially overcoming T cell exhaustion and enhanced proliferation and infiltration of CD8⁺ Tee cells. Immunomodulation by targeting GITR presents a promising opportunity in expanding the anticancer therapeutic options and can be considered for combination therapies with other immunomodulatory antibodies.

### BRIEF SUMMARY OF THE INVENTION

In one general aspect, the invention relates to isolated monoclonal antibodies or antigen-binding fragments thereof that specifically bind glucocorticoid-induced tumor necrosis factor receptor related protein (GITR), preferably human GITR.

Provided are isolated monoclonal antibodies or antigen-binding fragments thereof comprising a heavy chain complementarity determining region 1 (HCDR1), HCDR2, HCDR3, a light chain complementarity determining region 1 (LCDR1), LCDR2, and LCDR3, having the polypeptide sequences of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NO: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113,114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
   or a variant thereof comprising up to about three amino acid substitutions (e.g., one, two, or three amino acid substitutions) in the HCDR1, HCDR2, HCDR3, LCDR1, LCDR2, and/or LCDR3,
   wherein the antibody or antigen-binding fragment thereof specifically binds GITR, preferably human GITR.

In certain embodiments, the isolated monoclonal antibody or antigen-binding fragment thereof comprises a heavy chain variable region having a polypeptide sequence at least 95%, at least 96%, at least 97%, at least 98%, or at least 99% identical to SEQ ID NO: 1, 3, 5, 6, 8, 10, 12, 14, 16, 18, 19, 22, 24, 26, 28, 30, or 34, or a light chain variable region having a polypeptide sequence at least 95%, at least 96%, at least 97%, at least 98%, or at least 99% identical to SEQ ID NO:2, 4, 7, 9, 11, 13, 15, 17, 20, 21, 23, 25, 27, 29, 31, 32, 33, or 35.

In certain embodiments, the isolated monoclonal antibody or antigen-binding fragment thereof comprises:
is a. a heavy chain variable region having the polypeptide sequence of SEQ ID NO:1, and a light chain variable region having the polypeptide sequence of SEQ ID NO:2;
b. a heavy chain variable region having the polypeptide sequence of SEQ ID NO:3, and a light chain variable region having the polypeptide sequence of SEQ ID NO:4;
c. a heavy chain variable region having the polypeptide sequence of SEQ ID NO:5, and a light chain variable region having the polypeptide sequence of SEQ ID NO:7;
d. a heavy chain variable region having the polypeptide sequence of SEQ ID NO:6, and a light chain variable region having the polypeptide sequence of SEQ ID NO:7;
e. a heavy chain variable region having the polypeptide sequence of SEQ ID NO:8, and a light chain variable region having the polypeptide sequence of SEQ ID NO:9;
f. a heavy chain variable region having the polypeptide sequence of SEQ ID NO:10, and a light chain variable region having the polypeptide sequence of SEQ ID NO:11;
g, a heavy chain variable region having the polypeptide sequence of SEQ ID NO:12, and a light chain variable region having the polypeptide sequence of SEQ ID NO:13;
h. a heavy chain variable region having the polypeptide sequence of SEQ ID NO:14, and a light chain variable region having the polypeptide sequence of SEQ ID NO:15;
i. a heavy chain variable region having the polypeptide sequence of SEQ ID NO:16, and a light chain variable region having the polypeptide sequence of SEQ ID NO:17;
j. a heavy chain variable region having the polypeptide sequence of SEQ ID NO:18, and a light chain variable region having the polypeptide sequence of SEQ ID NO:20;
k. a heavy chain variable region having the polypeptide sequence of SEQ ID NO:18, and a light chain variable region having the polypeptide sequence of SEQ ID NO:21;
l. a heavy chain variable region having the polypeptide sequence of SEQ ID NO:19, and a light chain variable region having the polypeptide sequence of SEQ ID NO:20;
m. a heavy chain variable region having the polypeptide sequence of SEQ ID NO:19, and a light chain variable region having the polypeptide sequence of SEQ ID NO:21;
n. a heavy chain variable region having the polypeptide sequence of SEQ ID NO:22, and a light chain variable region having the polypeptide sequence of SEQ ID NO:23;
o. a heavy chain variable region having the polypeptide sequence of SEQ ID NO:24, and a light chain variable region having the polypeptide sequence of SEQ ID NO:25;
p. a heavy chain variable region having the polypeptide sequence of SEQ ID NO:26, and a light chain variable region having the polypeptide sequence of SEQ ID NO:27;
q. a heavy chain variable region having the polypeptide sequence of SEQ ID NO:28, and a light chain variable region having the polypeptide sequence of SEQ ID NO:29;
r. a heavy chain variable region having the polypeptide sequence of SEQ ID NO:30, and a light chain variable region having the polypeptide sequence of SEQ ID NO:31;
s. a heavy chain variable region having the polypeptide sequence of SEQ ID NO:30, and a light chain variable region having the polypeptide sequence of SEQ ID NO:32;
t. a heavy chain variable region having the polypeptide sequence of SEQ ID NO:30, and a light chain variable region having the polypeptide sequence of SEQ ID NO:33; or
u. a heavy chain variable region having the polypeptide sequence of SEQ ID NO:34, and a light chain variable region having the polypeptide sequence of SEQ ID NO:35.

In certain embodiments, the isolated monoclonal antibody or antigen-binding fragment thereof is chimeric. In certain embodiments, the isolated monoclonal antibody or antigen-binding fragment thereof is chimeric, and the isolated monoclonal antibody or antigen-binding fragment thereof comprises a human IgG1 constant regions or variants thereof. The variants of human IgG1 constant regions, for example, comprise at least one amino acid modification(s) selected from K214R, D356E, L358M and ΔK447.

In certain embodiments, the isolated monoclonal antibody or antigen-binding fragment thereof is human or humanized.

In certain embodiments, the isolated monoclonal antibody or antigen-binding fragment thereof is humanized, and the isolated monoclonal antibody or antigen-binding fragment thereof comprises:
a. a heavy chain variable region having the polypeptide sequence of SEQ ID NO:143, and a light chain variable region having the polypeptide sequence of SEQ ID NO:146;
b. a heavy chain variable region having the polypeptide sequence of SEQ ID NO:143, and a light chain variable region having the polypeptide sequence of SEQ ID NO:147;
c. a heavy chain variable region having the polypeptide sequence of SEQ ID NO:144, and a light chain variable region having the polypeptide sequence of SEQ ID NO:146;
d. a heavy chain variable region having the polypeptide sequence of SEQ ID NO:144, and a light chain variable region having the polypeptide sequence of SEQ ID NO:147;
e. a heavy chain variable region having the polypeptide sequence of SEQ ID NO:145, and a light chain variable region having the polypeptide sequence of SEQ ID NO:146;
f, a heavy chain variable region having the polypeptide sequence of SEQ ID NO:145, and a light chain variable region having the polypeptide sequence of SEQ ID NO:147;
g. a heavy chain variable region having the polypeptide sequence of SEQ ID NO:148, and a light chain variable region having the polypeptide sequence of SEQ ID NO:151;
h. a heavy chain variable region having the polypeptide sequence of SEQ ID NO:149, and a light chain variable region having the polypeptide sequence of SEQ ID NO:151;
i. a heavy chain variable region having the polypeptide sequence of SEQ ID NO:150, and a light chain variable region having the polypeptide sequence of SEQ ID NO:151;
j. a heavy chain variable region having the polypeptide sequence of SEQ ID NO:152, and a light chain variable region having the polypeptide sequence of SEQ ID NO:154; or
k. a heavy chain variable region having the polypeptide sequence of SEQ ID NO:1533 and a light chain variable region having the polypeptide sequence of SEQ ID NO:154.

In certain embodiments, the isolated monoclonal antibody or antigen-binding fragment thereof binds to GITR and is capable of inducing effector-mediated tumor cell lysis.

Also provided are isolated nucleic acids encoding the monoclonal antibodies or antigen-binding fragments thereof of the invention.

Also provided are vectors comprising the isolated nucleic acids encoding the monoclonal antibodies or antigen-binding fragments thereof of the invention.

Also provided are host cells comprising the vectors comprising the isolated nucleic acids encoding the monoclonal antibodies or antigen-binding fragments thereof of the invention.

In certain embodiments, provided is a pharmaceutical composition comprising the isolated monoclonal antibody or antigen-binding fragment thereof of the invention and a pharmaceutically acceptable carrier.

Also provided are methods of treating cancer in a subject in need thereof, comprising administering to the subject the pharmaceutical compositions of the invention. The cancer can be any liquid or solid cancer, for example, it can be selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of producing the monoclonal antibody or antigen-binding fragment thereof of the invention. The methods comprise culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or culture.

Also provided are methods of producing a pharmaceutical composition comprising the monoclonal antibody or antigen-binding fragment of the invention. The methods comprise combining the monoclonal antibody or antigen-binding fragment with a pharmaceutically acceptable carrier to obtain the pharmaceutical composition.

Also provided are methods of determining a level of GITR in a subject. The methods comprise (a) obtaining a sample from the subject; (b) contacting the sample with an isolated monoclonal antibody or antigen-binding fragment thereof of the invention; and (c) determining a level of GITR in the subject. The sample can, for example, be a tissue sample or a blood sample. The tissue sample can, for example, be a cancer tissue sample.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1K show the results of an antigen specific humoral response. The results demonstrate the anti-sera titer utilizing an ELISA-based assay for isotype control (FIG. 1A); pre-serum (FIG. 1B); PBS (FIG. 1C); mouse AD94 (FIG. 1D); mouse AD95 (FIG. 1E); mouse AD96 (FIG. 1F); mouse AD97 (FIG. 1G); mouse AD98 (FIG. 1H); mouse AD99 (FIG. 1I); mouse AD100 (FIG. 1J); and mouse AD101 (FIG. 1K).

FIGS. 2A-2D show graphs demonstrating the FACS results of anti-GITR mouse antibodies.

FIG. 3 shows a graph demonstrating the results of the anti-GITR mouse antibodies functional reporter gene dose response assay.

FIGS. 4A-4C show graphs demonstrating the FACS results utilizing both human GITR/Cho-K1 and cyno-GITR/Cho-K1 cell lines. FIGS. 4A and 4B show graphs demonstrating FACS binding of human GITR. FIG. 4C shows a graph demonstrating FACS binding of cyno-GITR.

FIGS. 5A-5C show graphs demonstrating the results of a FACS binding assay for humanized anti-GITR antibodies (FIG. 5A: 45F1F3; FIG. 5B: 270C5C10; and FIG. 5C 225H7D12).

FIGS. 6A-6B show graphs demonstrating the results of the humanized anti-GITR antibodies reporter gene functional bioassay.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fill within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-GITR antibodies and polynucleotides that encode them, GITR polypeptides and GITR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally. Current Protocols in Molecular Biology, F. M. Ausubel et al., eds., Current Protocols, a joint venture between Greene Publishing Associates, Inc. and John Wiley & Sons, Inc., (1995 Supplement)(Ausubel)).

Examples of algorithms that are suitable for determining percent sequence identity and sequence similarity am the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul. Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation of single- and double-stranded DNA, and DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Peptides, polypeptides, and proteins of the invention can, for example, comprise one or more amino acid substitutions.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

The term “amino acid substitution,” as used herein, refers to the replacement of one amino acid residue with another in a polypeptide sequence. A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “variant” as used herein in relation to an antibody or antigen binding fragment having a polypeptide with particular sequence features (the “reference antibody”) refers to a different antibody having a polypeptide comprising one or more (such as, for example, about 1 to about 25, about 1 to about 20, about 1 to about 15, about 1 to about 10, or about 1 to about 5) amino acid sequence substitutions, deletions, and/or additions as compared to the reference binding moiety. An anti-GITR antibody or antigen binding fragment variant at least retains specific binding to GITR. In some embodiments, anti-GITR antibody or antigen binding fragment variant can result from one or more (such as, for example, about 1 to about 25, about 1 to about 20, about 1 to about 15, about 1 to about 10, or about 1 to about 5) changes to an amino acid sequence of a reference antibody. In certain embodiments, the anti-GITR antibody or antigen binding fragment variant can comprise at least three (3) amino acid substitutions.

Antibodies

The invention generally relates to isolated anti-glucocorticoid-induced tumor necrosis factor receptor related protein (GITR) antibodies, nucleic acids and expression vectors encoding the antibodies, recombinant cells containing the vectors, and compositions comprising the antibodies. Methods of making the antibodies, and methods of using the antibodies to treat diseases including cancer are also provided. The antibodies of the invention possess one or more desirable functional properties, including but not limited to high-affinity binding to GITR, high specificity to GITR, and the ability to inhibit tumor growth in subjects in need thereof and in animal models when administered alone or in combination with other anti-cancer therapies.

In a general aspect, the invention relates to isolated monoclonal antibodies or antigen-binding fragments thereof that specifically bind GITR.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to GITR is substantially free of antibodies that do not bind to GITR). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)₂, an Fv fragment, a disulfide stabilized Fv fragment (dsFv), a (dsFv)₂, a bispecific dsFv (dsFv-dsFv), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′)₂.

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species, the variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on GITR and the second epitope is located on immune checkpoint molecules and/or other tumor associated immune suppressors or surface antigens.

As used herein, the term “GITR” refers to glucocorticoid-induced tumor necrosis factor receptor related protein. GITR belongs to the tumor necrosis factor receptor superfamily (TNFRSF), which are cytokine receptors that bind to tumor necrosis factors (TNFs) via an extracellular cysteine-rich domain. TNFRSF are involved in diverse cellular processes from apoptosis and inflammation to other signal transduction pathways in proliferation, survival, and differentiation. GITR was identified as a member of the TNFRSF, which protects T cells from apoptosis upon the exposure to glucocorticoid hormone (GC), a potent inducer of the death of T cells and commonly used as an immunosuppressive and anti-inflammatory agent. GITR is constitutively and exclusively expressed at high levels on CD25⁺CD4⁺ regulatory T cells. Its ligand, GITRL is predominantly expressed by activated antigen presenting cells (APCs). The expression of GITR and GITRL has also been noted on epidermal keratinocytes, osteoclast precursors, and epithelial cells. This suggests that the function of GITR and GITRL may not be limited to regulating immune responses but may also involve mediating leukocyte adhesion and migration. The anticancer therapeutic potential in modulating GITR function is attributed to its costimulatory role in effector T cells (T_{eff}) and the inhibition or depletion of tumor infiltrating regulatory T (T_{reg}) cells. Thus, GITR is a tumor-associated/tumor-specific antigen and anti-GITR monoclonal antibodies (mAbs) can be potential anti-cancer therapies. Further, GITR can be used to specifically target therapeutic molecules to cancer cells. An exemplary amino acid sequence of a human GITR is represented by GenBank Accession No. NP_004186.1 (Isoform 1), GenBank Accession No. NP_683699.1 (Isoform 2), and/or GenBank Accession No. NP_683700.1 (Isoform 3).

As used herein, an antibody that “specifically binds to GITR” refers to an antibody that binds to a GITR, preferably a human GITR, with a K_{D} of 1×10⁻⁷ M or less, preferably 1×10⁻⁸ M or less, more preferably 5×10⁻⁹M or less, 1×10⁻⁹ M or less, 5×10⁻¹⁰ M or less, or 1×10⁻¹⁰ M or less. The term “K_{D}” refers to the dissociation constant, which is obtained from the ratio of Kd to Ka (i.e., Kd/Ka) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a Biacore® system, or by using bio-layer interferometry technology, such as an Octet RED96 system.

The smaller the value of the K_{D} of an antibody, the higher affinity that the antibody binds to a target antigen.

According to a particular aspect, the invention relates to an isolated monoclonal antibody or antigen-binding fragment thereof comprising a heavy chain complementarity determining region 1 (HCDR1), a HCDR2, a HCDR3, a light chain complementarity determining region 1 (LCDR1), a LCDR2, and a LCDR3, having the polypeptide sequences of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
   or a variant thereof comprising up to about three (e.g., one, two or three) amino acid substitutions in the HCDR1, HCDR2, HCDR3, LCDR1, LCDR2, and/or LCDR3, wherein the antibody or antigen-binding fragment thereof specifically binds GITR, preferably human GITR

In some embodiments, the isolated monoclonal antibody or antigen-binding fragment thereof comprising a heavy chain complementarity determining region 1 (HCDR1), a HCDR2, a HCDR3, a light chain complementarity determining region 1 (LCDR1), a LCDR2, and a LCDR3, having the polypeptide sequences of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
   wherein the antibody or antigen-binding fragment thereof specifically binds GITR, preferably human GITR.

According to another particular aspect, the invention relates to an isolated monoclonal antibody or antigen-binding fragment thereof comprising a heavy chain variable region having a polypeptide sequence at least 85%, preferably 90%, more preferably 95% or more, such as 95%, 96%, 97%, 98%, or 99% identical to one of SEQ ID NOs: 1, 3, 5, 6, 8, 10, 12, 14, 16, 18, 19, 22, 24, 26, 28, 30, or 34, or a light chain variable region having a polypeptide sequence at least 85%, preferably 90%, more preferably 95% or more, such as 95%, 96%, 97%, 98%, or 99% identical to one of SEQ ID NOs: 2, 4, 7, 9, 11, 13, 15, 17, 20, 21, 23, 25, 27, 29, 31, 32, 33, or 35. According to one preferred embodiment, the isolated monoclonal antibody or antigen-binding fragment thereof of the invention comprises a heavy chain variable region having the polypeptide sequence at least 85%, preferably 90%, more preferably 95% or more, such as 95%, 96%, 97%, 98%, or 99% K identical to SEQ ID NO: 1, 3, 5, 6, 8, 10, 12, 14, 16, 18, 19, 22, 24, 26, 28, 30, or 34, and a light chain variable region having a polypeptide sequence at least 85%, preferably 90%, more preferably 95% or more, such as 95%, 96%, 97%, 98%, or 99% identical to SEQ ID NO: 2, 4, 7, 9, 11, 13, 15, 17, 20, 21, 23, 25, 27, 29, 31, 32, 33, or 35, respectively.

According to another particular aspect, the invention relates to an isolated monoclonal antibody or antigen-binding fragment thereof of the invention, comprising:
a. a heavy chain variable region having the polypeptide sequence of SEQ ID NO:1, and a light chain variable region having the polypeptide sequence of SEQ ID NO:2;
b. a heavy chain variable region having the polypeptide sequence of SEQ ID NO:3, and a light chain variable region having the polypeptide sequence of SEQ ID NO:4;
c. a heavy chain variable region having the polypeptide sequence of SEQ ID NO:5, and a light chain variable region having the polypeptide sequence of SEQ ID NO:7;
d. a heavy chain variable region having the polypeptide sequence of SEQ ID NO:6, and a light chain variable region having the polypeptide sequence of SEQ ID NO:7;
e. a heavy chain variable region having the polypeptide sequence of SEQ ID NO:8, and a light chain variable region having the polypeptide sequence of SEQ ID NO:9;
f. a heavy chain variable region having the polypeptide sequence of SEQ ID NO:10, and a light chain variable region having the polypeptide sequence of SEQ ID NO:11;
g. a heavy chain variable region having the polypeptide sequence of SEQ ID NO:12, and a light chain variable region having the polypeptide sequence of SEQ ID NO:13.
h. a heavy chain variable region having the polypeptide sequence of SEQ ID NO:14, and a light chain variable region having the polypeptide sequence of SEQ ID NO:15;
i. a heavy chain variable region having the polypeptide sequence of SEQ ID NO:16, and a light chain variable region having the polypeptide sequence of SEQ ID NO:17;
j. a heavy chain variable region having the polypeptide sequence of SEQ ID NO:18, and a light chain variable region having the polypeptide sequence of SEQ ID NO:20;
k. a heavy chain variable region having the polypeptide sequence of SEQ ID NO:18, and a light chain variable region having the polypeptide sequence of SEQ ID NO:21;
l. a heavy chain variable region having the polypeptide sequence of SEQ ID NO:19, and a light chain variable region having the polypeptide sequence of SEQ ID NO:20;
m. a heavy chain variable region having the polypeptide sequence of SEQ ID NO:19, and a light chain variable region having the polypeptide sequence of SEQ ID NO:21;
n. a heavy chain variable region having the polypeptide sequence of SEQ ID NO:22, and a light chain variable region having the polypeptide sequence of SEQ ID NO:23;
o. a heavy chain variable region having the polypeptide sequence of SEQ ID NO:24, and a light chain variable region having the polypeptide sequence of SEQ ID NO:25;
p. a heavy chain variable region having the polypeptide sequence of SEQ ID NO:26, and a light chain variable region having the polypeptide sequence of SEQ ID NO:27;
q. a heavy chain variable region having the polypeptide sequence of SEQ ID NO:28, and a light chain variable region having the polypeptide sequence of SEQ ID NO:29;
r. a heavy chain variable region having the polypeptide sequence of SEQ ID NO:30, and a light chain variable region having the polypeptide sequence of SEQ ID NO:31;
s. a heavy chain variable region having the polypeptide sequence of SEQ ID NO:30, and a light chain variable region having the polypeptide sequence of SEQ ID NO:32;
t. a heavy chain variable region having the polypeptide sequence of SEQ ID NO:30, and a light chain variable region having the polypeptide sequence of SEQ ID NO:33; or
u. a heavy chain variable region having the polypeptide sequence of SEQ ID NO:34, and a light chain variable region having the polypeptide sequence of SEQ ID NO:35.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 36, 37, 38, 39, 40, and 41, respectively. In another embodiment, the isolated monoclonal antibody or antigen-binding fragment thereof comprises a heavy chain variable region having a polypeptide sequence at least 85%, preferably 90%, more preferably 93% or more, such as 95%, 96%, 97%, 98%, or 99% identical to SEQ ID NO:1, and a light chain variable region having a polypeptide sequence at least 85%, preferably 90%, more preferably 95% or more, such as 95%, 96%, 97%, 98%, or 99% identical to SEQ ID NO:2. Preferably, the isolated monoclonal antibody or antigen-binding fragment thereof comprises a heavy chain variable region having the polypeptide sequence of SEQ ID NO:1; and a light chain variable region having the polypeptide sequence of SEQ ID NO:2.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 42, 43, 44, 45, 46, and 47, respectively, in another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:3, and a light chain variable region having a polypeptide sequence at least 85%, preferably 90%, more preferably 95% or more, such as 95%, 96%, 97%, 98%, or 99% identical to SEQ ID NO:4. Preferably, the isolated monoclonal antibody or antigen-binding fragment thereof comprises a heavy chain variable region having the polypeptide sequence of SEQ ID NO:3, and a light chain variable region having the polypeptide sequence of SEQ ID NO:4.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 48, 49, 50, 54, 55, and 56, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:5, and a light chain variable region having a polypeptide sequence at least 85%, preferably 90%, more preferably 95% or more, such as 95%, 96%, 97%, 98%, or 99% identical to SEQ ID NO:7. Preferably, the isolated monoclonal antibody or antigen-binding fragment thereof comprises a heavy chain variable region having the polypeptide sequence of SEQ ID NO:5; and a light chain variable region having the polypeptide sequence of SEQ ID NO:7.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 51, 52, 53, 54, 55, and 56, respectively, in another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:6, and a light chain variable region having a polypeptide sequence at least 85%, preferably 90%, more preferably 95% or more, such as 95%, 96%, 97%, 98%, or 99% identical to SEQ ID NO:7. Preferably, the isolated monoclonal antibody or antigen-binding fragment thereof comprises a heavy chain variable region having the polypeptide sequence of SEQ ID NO:6; and a light chain variable region having the polypeptide sequence of SEQ ID NO:7.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 57, 58, 59, 60, 61, and 62, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97% 98%, or 99% identical to SEQ ID NO:8, and a light chain variable region having a polypeptide sequence at least 85%, preferably 90%, more preferably 95% or more, such as 95%, 96%, 97%, 98%, or 99% identical to SEQ ID NO:9. Preferably, the isolated monoclonal antibody or antigen-binding fragment thereof comprises a heavy chain variable region having the polypeptide sequence of SEQ ID NO:8; and a light chain variable region having the polypeptide sequence of SEQ ID NO:9.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 63, 64, 65, 66, 67, and 68, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:10, and a light chain variable region having a polypeptide sequence at least 85%, preferably 90%, more preferably 95% or more, such as 95%, 96%, 97%, 98%, or 99% identical to SEQ ID NO:11. Preferably, the isolated monoclonal antibody or antigen-binding fragment thereof comprises a heavy chain variable region having the polypeptide sequence of SEQ ID NO:10; and a light chain variable region having the polypeptide sequence of SEQ ID NO:11.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 69, 70, 71, 72, 73, and 74, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:12, and a light chain variable region having a polypeptide sequence at least 85%, preferably 90%, more preferably 95% or more, such as 95%, 96%, 97%, 98%, or 99% identical to SEQ ID NO:13. Preferably, the isolated monoclonal antibody or antigen-binding fragment thereof comprises a heavy chain variable region having the polypeptide sequence of SEQ ID NO:12, and a light chain variable region having the polypeptide sequence of SEQ ID NO:13.

In one embodiment, the invention relates to an isolated monoclonal antibody or antigen-binding fragment thereof, comprising HCDR1. HCDR2, HCDR3, LCDR1, LCDR2 and LCDR3, having the polypeptide sequences of SEQ ID NOs: 75, 76, 77.78, 79, and 80, respectively, in another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:14, and a light chain variable region having a polypeptide sequence at least 85%, preferably 90, more preferably 95% or more, such as 95%, 96%, 97%, 98%, or 99% identical to SEQ ID NO:15. Preferably, the isolated monoclonal antibody or antigen-binding fragment thereof comprises a heavy chain variable region having the polypeptide sequence of SEQ ID NO:14; and a light chain variable region having the polypeptide sequence of SEQ ID NO:15.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 81, 82, 83, 84, 85, and 86, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:16, and a light chain variable region having a polypeptide sequence at least 85%, preferably 90%, more preferably 95% or more, such as 95%/a, 96%, 97%, 98%, or 99% identical to SEQ ID NO:17. Preferably, the isolated monoclonal antibody or antigen-binding fragment thereof comprises a heavy chain variable region having the polypeptide sequence of SEQ ID NO:16; and a light chain variable region having the polypeptide sequence of SEQ ID NO:17.

In one embodiment, the invention relates to an isolated monoclonal antibody or antigen-binding fragment thereof, comprising HCDR1, HCDR2, HCDR3, LCDR1. LCDR2 and LCDR3, having the polypeptide sequences of SEQ ID NOs: 87, 88, 89, 93, 94, and 95, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:18, and a light chain variable region having a polypeptide sequence at least 85%, preferably 90%, more preferably 95% or more, such as 95%, 96%, 97%, 98%, or 99% identical to SEQ ID NO:20. Preferably, the isolated monoclonal antibody or antigen-binding fragment thereof comprises a heavy chain variable region having the polypeptide sequence of SEQ ID NO:18; and a light chain variable region having the polypeptide sequence of SEQ ID NO:20.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 87, 88, 89, 96, 97, and 98, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:18, and a light chain variable region having a polypeptide sequence at least 85%, preferably 90%, more preferably 95% or more, such as 95%, 96%, 97%, 98%, or 99% identical to SEQ ID NO:21. Preferably, the isolated monoclonal antibody or antigen-binding fragment thereof comprises a heavy chain variable region having the polypeptide sequence of SEQ ID NO:18; and a light chain variable region having the polypeptide sequence of SEQ ID NO:21.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 90, 91, 92, 93, 94, and 95, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:19, and a light chain variable region having a polypeptide sequence at least 85%, preferably 90%, more preferably 95% or more, such as 95%, 96%, 97%, 98%, or 99% identical to SEQ ID NO:20. Preferably, the isolated monoclonal antibody or antigen-binding fragment thereof comprises a heavy chain variable region having the polypeptide sequence of SEQ ID NO:19; and a light chain variable region having the polypeptide sequence of SEQ ID NO:20.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 90, 91, 92, 96, 97, and 98, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:19, and a light chain variable region having a polypeptide sequence at least 85%, preferably 90%, more preferably 95% or more, such as 95%, 96%, 97%, 98%, or 99% identical to SEQ ID NO:21. Preferably, the isolated monoclonal antibody or antigen-binding fragment thereof comprises a heavy chain variable region having the polypeptide sequence of SEQ ID NO:19; and a light chain variable region having the polypeptide sequence of SEQ ID NO:21.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 99, 100, 101, 102, 103, and 104, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:22, and a light chain variable region having a polypeptide sequence at least 85%, preferably 90%, more preferably 95% or more, such as 95%, 96%, 97%, 98%, or 99% identical to SEQ ID NO:23. Preferably, the isolated monoclonal antibody or antigen-binding fragment thereof comprises a heavy chain variable region having the polypeptide sequence of SEQ ID NO:22; and a light chain variable region having the polypeptide sequence of SEQ ID NO:23.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 105, 106, 107, 108, 109, and 110, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:24, and a light chain variable region having a polypeptide sequence at least 85%, preferably 90%, more preferably 95% or more, such as 95%, 96%, 97%, 98%, or 99% identical to SEQ ID NO:25. Preferably, the isolated monoclonal antibody or antigen-binding fragment thereof comprises a heavy chain variable region having the polypeptide sequence of SEQ ID NO:24; and a light chain variable region having the polypeptide sequence of SEQ ID NO:25.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 11, 112, 113, 114, 115, and 116, respectively. In another embodiment, the isolated monoclonal antibody or antigen-binding fragment thereof comprises a heavy chain variable region having a polypeptide sequence at least 85%, preferably 90%, more preferably 95% or more, such as 95%, %%, 97%, 98%, or 99% identical to SEQ ID NO:26, and a light chain variable region having a polypeptide sequence at least 85%, preferably 90%, more preferably 95% or more, such as 95%, 96%, 97%, 98%, or 99% identical to SEQ ID NO:27. Preferably, the isolated monoclonal antibody or antigen-binding fragment thereof comprises a heavy chain variable region having the polypeptide sequence of SEQ ID NO:26; and a light chain variable region having the polypeptide sequence of SEQ ID NO:27.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 117, 118,119, 120, 121, and 122, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:28, and a light chain variable region having a polypeptide sequence at least 85%, preferably 90%, more preferably 95% or more, such as 95%, 96%, 97%, 98%, or 99% identical to SEQ ID NO:29. Preferably, the isolated monoclonal antibody or antigen-binding fragment thereof comprises a heavy chain variable region having the polypeptide sequence of SEQ ID NO:28; and a light chain variable region having the polypeptide sequence of SEQ ID NO:29.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 123, 124, 125, 126, 127, and 128, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:30, and a light chain variable region having a polypeptide sequence at least 85%, preferably 90%, more preferably 95% or more, such as 95%, 96%, 97%, 98, or 99/identical to SEQ ID NO:31. Preferably, the isolated monoclonal antibody or antigen-binding fragment thereof comprises a heavy chain variable region having the polypeptide sequence of SEQ ID NO:30, and a light chain variable region having the polypeptide sequence of SEQ ID NO:31.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 123, 124, 125, 129, 130, and 131, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:30, and a light chain variable region having a polypeptide sequence at least 85%, preferably 90%, more preferably 95% or more, such as 95%, 96%, 97%, 98%, or 99% identical to SEQ ID NO:32. Preferably, the isolated monoclonal antibody or antigen-binding fragment thereof comprises a heavy chain variable region having the polypeptide sequence of SEQ ID NO:30; and a light chain variable region having the polypeptide sequence of SEQ ID NO:32.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 123, 124, 125, 132, 133, and 134, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:30, and a light chain variable region having a polypeptide sequence at least 85%, preferably 90%, more preferably 95% or more, such as 95%, 96%, 97%, 98%, or 99% identical to SEQ ID NO:33. Preferably, the isolated monoclonal antibody or antigen-binding fragment thereof comprises a heavy chain variable region having the polypeptide sequence of SEQ ID NO:30; and a light chain variable region having the polypeptide sequence of SEQ ID NO:33.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 135, 136, 137, 138, 139, and 140, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:34, and a light chain variable region having a polypeptide sequence at least 85%, preferably 90%, more preferably 95% or more, such as 95%, 96%, 97%, 98%, or 99% identical to SEQ ID NO:35. Preferably, the isolated monoclonal antibody or antigen-binding fragment thereof comprises a heavy chain variable region having the polypeptide sequence of SEQ ID NO:34; and a light chain variable region having the polypeptide sequence of SEQ ID NO:35.

According to another particular aspect, the invention relates to an isolated monoclonal antibody or antigen-binding fragment thereof, wherein the antibody or antigen-binding fragment thereof is chimeric. In certain embodiments, the isolated monoclonal antibody or antigen-binding fragment thereof is chimeric, and the isolated monoclonal antibody or antigen-binding fragment thereof comprises a human IgG1 constant regions or variants thereof. The human IgG1 constant regions comprise a human IgG1 heavy chain constant region and a light chain kappa constant region. The variants of human IgG1 heavy chain constant region, for example, comprise at least one amino acid modification selected from K214R, D356E, L358M, and ΔK447. In certain embodiments, the constant region of human IgG1 heavy chain comprises the polypeptide sequence of SEQ ID NO:141, and the constant region of human IgG1 light chain comprises the polypeptide sequence of SEQ ID NO:142. In certain embodiments, the constant region of the variant human IgG1 heavy chain comprises the polypeptide sequence of SEQ ID NO:155, and the constant region of human IgG1 light chain comprises the polypeptide sequence of SEQ ID NO:142. In certain embodiments, the constant region of the variant of human IgG1 heavy chain comprises the polypeptide sequence of SEQ ID NO:156, and the constant region of human IgG1 light chain comprises the polypeptide sequence of SEQ ID NO:142. By way of an example, the variable regions of clones 45F1F3, 225H7D12, 270C5C10, 163H12G7, 172H7B9, and 223F12C4, described below, were fused onto the human IgG1 constant regions with the modifications of K214R, D356E, L358M and ΔK447. By way of another example, variable regions of clones 204C10G12, 225A8D9, 274C7H2, 384H3H11, 134D7B3, 177C6811, 215F2A5, and 223H11H1, described below, were fused onto human IgG1 constant regions with the modification of K214R.

In some embodiments, the anti-GITR monoclonal antibody or antigen-binding fragment thereof is a chimeric monoclonal antibody or antigen-binding fragment thereof. Certain chimeric antibodies are described, e.g., in U.S. Pat. No. 4,816,567; and Morrison et al., Proc. Natl. Acad. Si. USA, 81:6851-6855 (1984)). In one example, a chimeric antibody comprises a non-human variable region (e.g., a variable region derived from a murine, such as mouse) and a human constant region. In a further example, a chimeric antibody is a “class switched” antibody in which the class or subclass has been changed from that of the parent antibody.

According to another particular aspect, the invention relates to an isolated monoclonal antibody or antigen-binding fragment thereof, wherein the antibody or antigen-binding fragment thereof is human or humanized.

In some embodiments, a chimeric antibody or antigen-binding fragment thereof is a humanized antibody or antigen-binding fragment thereof. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991)(describing “resurfacing”); Dall'Acqua et al., Methods 36:43-60 (2005)(describing “FR shuffling”); and Osbourn et al., Methods 36:61-68 (2005) and Klimka et al., Br. J. Cancer, 83:252-260 (2000)(describing the “guided selection” approach to FR shuffling).

Human framework regions that can be used for humanization include but are not limited to: framework regions selected using the “best-fit” method (see, e.g., Sims et al. J Immunol. 151:22% (1993)); framework regions derived from the consensus sequence of human antibodies of a particular subgroup of light or heavy chain variable regions (see, e.g., Carter et al. Proc. Natl. Acad Si. USA, 89:4285 (1992); and Presta et al. J Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, the isolated monoclonal antibody or antigen-binding fragment thereof is humanized, and the isolated monoclonal antibody or antigen-binding fragment thereof comprises:
a. a heavy chain variable region having the polypeptide sequence of SEQ ID NO:143, and a light chain variable region having the polypeptide sequence of SEQ ID NO:146;
b. a heavy chain variable region having the polypeptide sequence of SEQ ID NO:143, and a light chain variable region having the polypeptide sequence of SEQ ID NO:147;
c. a heavy chain variable region having the polypeptide sequence of SEQ ID NO:144, and a light chain variable region having the polypeptide sequence of SEQ ID NO:146;
d. a heavy chain variable region having the polypeptide sequence of SEQ ID NO:144, and a light chain variable region having the polypeptide sequence of SEQ ID NO:147;
e. a heavy chain variable region having the polypeptide sequence of SEQ ID NO:145, and a light chain variable region having the polypeptide sequence of SEQ ID NO:146;
f. a heavy chain variable region having the polypeptide sequence of SEQ ID NO:145, and a light chain variable region having the polypeptide sequence of SEQ ID NO:147;
g. a heavy chain variable region having the polypeptide sequence of SEQ ID NO:148, and a light chain variable region having the polypeptide sequence of SEQ ID NO:151;
h. a heavy chain variable region having the polypeptide sequence of SEQ ID NO:149, and a light chain variable region having the polypeptide sequence of SEQ ID NO:151;
i. a heavy chain variable region having the polypeptide sequence of SEQ ID NO:150, and a light chain variable region having the polypeptide sequence of SEQ ID NO:151;
j. a heavy chain variable region having the polypeptide sequence of SEQ ID NO:152, and a light chain variable region having the polypeptide sequence of SEQ ID NO:154; or
k. a heavy chain variable region having the polypeptide sequence of SEQ ID NO:153, and a light chain variable region having the polypeptide sequence of SEQ ID NO:154.

In one embodiment, the invention relates to an isolated humanized monoclonal antibody or antigen-binding fragment thereof, comprising HCDR1, HCDR2, HCDR3, LCDR1, LCDR2 and LCDR3, having the polypeptide sequences of SEQ ID NOs: 36, 37, 38.39, 40, and 41, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NOs:143, 144 or 145 and a light chain variable region having a polypeptide sequence at least 85%, preferably 90%, more preferably 95% or more, such as 95%, 6%%, 97%, 98%, or 99% identical to SEQ ID NOs:146 or 147. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:143; and a light chain variable region having the polypeptide sequence at least 85%, 90%, 95%, 6%, 97%, 98% or 99% identical to SEQ ID NO:146. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:143; and a light chain variable region having the polypeptide sequence at least 85%, 90%, 95%, 96%, 97, 98% or 99% identical to SEQ ID NO:147. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:144; and a light chain variable region having the polypeptide sequence at least 85%, 90%, 95%, 6%%, 97%, 98% or 99% identical to SEQ ID NO:146. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:144; and a light chain variable region having the polypeptide sequence at least 85%, 90%, 95%, 96%, 97%, 98% or 99% identical to SEQ ID NO:147. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:145; and a light chain variable region having the polypeptide sequence at least 85%, 90%, 95%, 96%, 97%, 98% or 99% identical to SEQ ID NO:146. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:145; and a light chain variable region having the polypeptide sequence at least 85%, 90%, 95%, 96%, 97%, 98% or 99% identical to SEQ ID NO:147.

In one embodiment, the invention relates to an isolated humanized monoclonal antibody or antigen-binding fragment thereof, comprising HCDR1, HCDR2, HCDR3, LCDR1, LCDR2 and LCDR3, having the polypeptide sequences of SEQ ID NOs: 42, 43, 44, 45, 46, and 47, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NOs:148, 149 or 150 and a light chain variable region having a polypeptide sequence at least 85%, preferably 90%, more preferably 95% or more, such as 95%, 96%, 97%, 98%, or 99% identical to SEQ ID NO:151. Preferably, the isolated humanized monoclonal antibody or antigen-binding fragment thereof comprises a heavy chain variable region having the polypeptide sequence at least 85%, 90%, 95, 96%, 97%, 98% or 99% identical to SEQ ID NO:148; and a light chain variable region having the polypeptide sequence at least 85%, 90%, 95%, 96%, 97%, 98% or 99% identical to SEQ ID NO:151. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:149; and a light chain variable region having the polypeptide sequence at least 85%, 90%, 95%, 96%, 97%, 98% or 99% identical to SEQ ID NO:151. Preferably, the isolated humanized monoclonal antibody or antigen-binding fragment thereof comprises a heavy chain variable region having the polypeptide sequence at least 85%, 90%, 95%, 9%, 97%, 98% or 99% identical to SEQ ID NO:150; and a light chain variable region having the polypeptide sequence at least 85%, 90%, 95%, 96%, 97%, 98% or 99% identical to SEQ ID NO:151.

In one embodiment, the invention relates to an isolated humanized monoclonal antibody or antigen-binding fragment thereof, comprising HCDR1, HCDR2, HCDR3, LCDR1, LCDR2 and LCDR3, having the polypeptide sequences of SEQ ID NOs: 48, 49, 50, 54, 55, and 56, respectively. In another embodiment, the isolated humanized monoclonal antibody or antigen-binding fragment thereof comprises a heavy chain variable region having a polypeptide sequence at least 85/preferably 90%, more preferably 95% or more, such as 95%, 96%, 97%, 98%, or 99% identical to SEQ ID NOs:152 or 153 and a light chain variable region having a polypeptide sequence at least 85%, preferably 90%, more preferably 95% or more, such as 95%, 96%, 97%, 98%, or 99% identical to SEQ ID NO:154. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:152; and a light chain variable region having the polypeptide sequence at least 85%, 90, 95%, 96%, 97%, 98% or 99% identical to SEQ ID NO:154. Preferably, the isolated humanized monoclonal antibody or antigen-binding fragment thereof comprises a heavy chain variable region having the polypeptide sequence at least 85%, 90%, 95%, 96%, 97%, 98% or 99% identical to SEQ ID NO:153; and a light chain variable region having the polypeptide sequence at least 85%, 90%, 95%, 96%, 97%, 98% or 99% identical to SEQ ID NO:154.

The CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a Kabat.

In another general aspect, the invention relates to an isolated nucleic acid encoding a monoclonal antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding a monoclonal antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques am well known to those skilled in the art in view of the present disclosure.

In another general aspect, the invention relates to a host cell comprising an isolated nucleic acid encoding a monoclonal antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal antibody or antigen-binding fragment thereof of the invention, comprising culturing a cell comprising a nucleic acid encoding the monoclonal antibody or antigen-binding fragment thereof under conditions to produce a monoclonal antibody or antigen-binding fragment thereof of the invention, and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

Pharmaceutical Compositions

In another general aspect, the invention relates to a pharmaceutical composition, comprising an isolated monoclonal antibody or antigen-binding fragment thereof of the invention and a pharmaceutically acceptable carrier. The term “pharmaceutical composition” as used herein means a product comprising an antibody of the invention together with a pharmaceutically acceptable carrier. Antibodies of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

In another embodiment of the invention, the pharmaceutical composition comprises a buffer. In another embodiment of the invention, the pharmaceutical composition comprises a preservative. In another embodiment of the invention, the pharmaceutical composition comprises an isotonic agent. In another embodiment of the invention, the pharmaceutical composition comprises a chelating agent. In another embodiment of the invention, the pharmaceutical composition comprises a stabilizer.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants.

In a further embodiment of the invention, the pharmaceutical composition comprises one or more protease inhibitors, such as, e.g., EDTA, and/or benzamidine hydrochloric acid (HCl).

In another general aspect, the invention relates to a method of producing a pharmaceutical composition comprising a monoclonal antibody or antigen-binding fragment thereof of the invention, comprising combining a monoclonal antibody or antigen-binding fragment thereof with a pharmaceutically acceptable carrier to obtain the pharmaceutical composition.

Methods of Use

In another general aspect, the invention relates to a method of targeting GITR on a cancer cell surface in a subject, the method comprising administering to the subject an isolated monoclonal antibody or antigen binding fragment thereof that specifically binds GITR or a pharmaceutical composition of the invention. Binding of the monoclonal antibody or antigen-binding fragment thereof to GITR can mediate complement-dependent cytotoxicity (CDC), antibody-dependent phagocytosis (ADPC), and/or antibody-dependent cellular cytotoxicity (ADCC) or other effects that result in the death of the targeted cancer cell. The monoclonal antibody or antigen-binding fragment thereof can, for example, serve to recruit conjugated drugs, and/or can form a bispecific antibody with another monoclonal antibody to mediate the death of the targeted cancer cell.

The functional activity of antibodies and antigen-binding fragments thereof that bind GITR can be characterized by methods known in the art and as described herein. Methods for characterizing antibodies and antigen-binding fragments thereof that bind GITR include, but are not limited to, affinity and specificity assays including Biacore, ELISA, and OctetRed analysis; binding assays to detect the binding of antibodies to GITR on cancer cells or cells recombinantly expressing GITR by FACS. According to particular embodiments, the methods for characterizing antibodies and antigen-binding fragments thereof that bind GITR include those described below.

In another general aspect, the invention relates to a method of treating a cancer in a subject in need thereof, comprising administering to the subject an isolated monoclonal antibody or antigen binding fragment thereof that specifically binds GITR or a pharmaceutical composition of the invention. The cancer can, for example, be selected from but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical composition comprises a therapeutically effective amount of an anti-GITR antibody or antigen-binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to anti-GITR antibodies or antigen-binding fragments thereof, a therapeutically effective amount means an amount of the anti-GITR antibody or antigen-binding fragment thereof that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating.” and “treatment,” can also refer to causing regression, preventing the progression, or at least slowing down the progression of the disease, disorder, or condition. In a particular embodiment, “treat,” “treating.” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the compositions can be used in combination with another treatment including, but not limited to, a chemotherapy, an anti-CD20 mAb, an anti-CD47 mAb, an anti-LAG-3 mAb, an anti-CD73 mAb, an anti-CTLA-4 mAb, an anti-PD-L1 mAb, an anti-PD-1 mAb, a PD-1/PD-L1 therapy, other immuno-oncology drugs, an antiangiogenic agent, a radiation therapy, an antibody-drug conjugate (ADC), a targeted therapy, or other anticancer drugs. Anti-GITR antibodies can be used to construct bispecific antibodies with partner mAbs against immune checkpoint molecules and/or other tumor surface antigens to treat cancers/tumors that express both GITR and the specific tumor associated antigen.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In another general aspect, the invention relates to a method of determining a level of GITR in a subject. The methods comprise (a) obtaining a sample from the subject; (b) contacting the sample with a monoclonal antibody or antigen-binding fragment thereof of the invention; and (c) determining a level of GITR in the subject.

As used herein, “sample” refers to a biological sample isolated from a subject and can include, but is not limited to, whole blood, serum, plasma, blood cells, endothelial cells, tissue biopsies (e.g., a cancer tissue), lymphatic fluid, ascites fluid, interstitial fluid, bone marrow, cerebrospinal fluid, saliva, mucous, sputum, sweat, urine, or any other secretion, excretion, or other bodily fluids. A “blood sample” refers to whole blood or any fraction thereof, including blood cells, serum, and plasma.

In certain embodiments, the level of GITR in the subject can be determined utilizing assays selected from, but not limited to, a Western blot assay, an ELISA assay, and/or an immunohistochemistry (IHC). Relative protein levels can be determined by utilizing Western blot analysis and immunohistochemistry (IHC), and absolute protein levels can be determined by utilizing an ELISA assay. When determining the relative levels of GITR, the levels of GITR can be determined between at least two samples, e.g., between samples from the same subject at different time points, between samples from different tissues in the same subject, and/or between samples from different subjects. Alternatively, when determining absolute levels of GITR, such as by an ELISA assay, the absolute level of GITR in the sample can be determined by creating a standard for the ELISA assay prior to testing the sample. A person skilled in the art would understand which analytical techniques to utilize to determine the level of GITR in a sample from the subject utilizing the antibodies or antigen-binding fragments thereof of the invention.

Utilizing methods of determining a level of GITR in a sample from a subject can lead to the diagnosis of abnormal (elevated, reduced, or insufficient) GITR levels in a disease and making appropriate therapeutic decisions. Such a disease can include, for example, cancer. Additionally, by monitoring the levels of GITR in a subject, the risk of developing a disease as indicated above can be determined based on the knowledge of the level of GITR in a particular disease and/or during the progression of the particular disease.

### Embodiments

This invention provides the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequence of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
   or a variant thereof comprising up to about three (e.g., one, two, or three) amino acid substitutions in HCDR1, HCDR2, HCDR3, LCDR1, LCDR2, and/or LCDR3, wherein the antibody or antigen-binding fragment thereof specifically binds glucocorticoid-induced tumor necrosis factor receptor related protein (GITR), preferably human GITR.

Embodiment 2 is the isolated monoclonal antibody or antigen-binding fragment thereof of embodiment 1, wherein the isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequence of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
   wherein the antibody or antigen-binding fragment thereof specifically binds glucocorticoid-induced tumor necrosis factor receptor related protein (GITR), preferably human GITR.

Embodiment 3 is the isolated monoclonal antibody or antigen-binding fragment thereof of embodiment 1 or 2, comprising a heavy chain variable region having a polypeptide sequence at least 95%, at least 96%, at least 97%, at least 98%, or at least 99/identical to SEQ ID NO: 1, 3, 5, 6, 8, 10, 12, 14, 16, 22, 24, 26, 28, 30, or 34, or a light chain variable region having a polypeptide sequence at least 95%, at least 96%, at least 97%, at least 98%, or at least 99% identical to SEQ ID NO: 2, 4, 7, 9, 11, 13, 15, 17, 23, 25, 27, 29, 31, 32, 33, or 35.

Embodiment 4 is the isolated monoclonal antibody or antigen-binding fragment thereof of embodiments 1 to 3, comprising:
is a. a heavy chain variable region having the polypeptide sequence of SEQ ID NO:1, and a light chain variable region having the polypeptide sequence of SEQ ID NO:2;
b. a heavy chain variable region having the polypeptide sequence of SEQ ID NO:3, and a light chain variable region having the polypeptide sequence of SEQ ID NO:4;
c. a heavy chain variable region having the polypeptide sequence of SEQ ID NO:5, and a light chain variable region having the polypeptide sequence of SEQ ID NO:7;
d. a heavy chain variable region having the polypeptide sequence of SEQ ID NO:6, and a light chain variable region having the polypeptide sequence of SEQ ID NO:7;
e. a heavy chain variable region having the polypeptide sequence of SEQ ID NO:8, and a light chain variable region having the polypeptide sequence of SEQ ID NO:9;
f. a heavy chain variable region having the polypeptide sequence of SEQ ID NO:10, and a light chain variable region having the polypeptide sequence of SEQ ID NO:11;
g, a heavy chain variable region having the polypeptide sequence of SEQ ID NO:12, and a light chain variable region having the polypeptide sequence of SEQ ID NO:13;
h. a heavy chain variable region having the polypeptide sequence of SEQ ID NO:14, and a light chain variable region having the polypeptide sequence of SEQ ID NO:15;
i. a heavy chain variable region having the polypeptide sequence of SEQ ID NO:16, and a light chain variable region having the polypeptide sequence of SEQ ID NO:17;
j. a heavy chain variable region having the polypeptide sequence of SEQ ID NO:22, and a light chain variable region having the polypeptide sequence of SEQ ID NO:23;
k. a heavy chain variable region having the polypeptide sequence of SEQ ID NO:24, and a light chain variable region having the polypeptide sequence of SEQ ID NO:25;
l. a heavy chain variable region having the polypeptide sequence of SEQ ID NO:26, and a light chain variable region having the polypeptide sequence of SEQ ID NO:27;
m. a heavy chain variable region having the polypeptide sequence of SEQ ID NO:28, and a light chain variable region having the polypeptide sequence of SEQ ID NO:29;
n. a heavy chain variable region having the polypeptide sequence of SEQ ID NO:30, and a light chain variable region having the polypeptide sequence of SEQ ID NO:31;
o. a heavy chain variable region having the polypeptide sequence of SEQ ID NO:30, and a light chain variable region having the polypeptide sequence of SEQ ID NO:32;
p. a heavy chain variable region having the polypeptide sequence of SEQ ID NO:30, and a light chain variable region having the polypeptide sequence of SEQ ID NO:33; or
q. a heavy chain variable region having the polypeptide sequence of SEQ ID NO:34, and a light chain variable region having the polypeptide sequence of SEQ ID NO:35.

Embodiment 5 is the isolated monoclonal antibody or antigen-binding fragment thereof of any one of embodiments 1 to 4, wherein the antibody or antigen-binding fragment thereof is chimeric.

Embodiment 6 is the isolated monoclonal antibody or antigen-binding fragment thereof of embodiment 5, wherein the monoclonal antibody or antigen-binding fragment thereof comprises the human IgG1 constant regions.

Embodiment 7 is the isolated monoclonal antibody or antigen-binding fragment thereof of embodiment 6, wherein the human IgG1 constant regions comprise at least one amino acid modification(s) selected from K214R, D356E, L358M, and ΔK447.

Embodiment 8 is the isolated monoclonal antibody or antigen-binding fragment thereof of any one of embodiments 5 to 7, wherein the antibody or antigen-binding fragment thereof is human or humanized.

Embodiment 9 is the isolated monoclonal antibody or antigen-binding fragment thereof of embodiment 8, comprising:
a. a heavy chain variable region having the polypeptide sequence of SEQ ID NO:143, and a light chain variable region having the polypeptide sequence of SEQ ID NO:146;
b. a heavy chain variable region having the polypeptide sequence of SEQ ID NO:143, and a light chain variable region having the polypeptide sequence of SEQ ID NO:147;
c. a heavy chain variable region having the polypeptide sequence of SEQ ID NO:144, and a light chain variable region having the polypeptide sequence of SEQ ID NO:146;
d. a heavy chain variable region having the polypeptide sequence of SEQ ID NO:144, and a light chain variable region having the polypeptide sequence of SEQ ID NO:147;
e. a heavy chain variable region having the polypeptide sequence of SEQ ID NO:145, and a light chain variable region having the polypeptide sequence of SEQ ID NO:146;
f. a heavy chain variable region having the polypeptide sequence of SEQ ID NO:145, and a light chain variable region having the polypeptide sequence of SEQ ID NO:147;
g. a heavy chain variable region having the polypeptide sequence of SEQ ID NO:148, and a light chain variable region having the polypeptide sequence of SEQ ID NO:151;
h. a heavy chain variable region having the polypeptide sequence of SEQ ID NO:149, and a light chain variable region having the polypeptide sequence of SEQ ID NO:151;
i. a heavy chain variable region having the polypeptide sequence of SEQ ID NO:150, and a light chain variable region having the polypeptide sequence of SEQ ID NO:151;
j. a heavy chain variable region having the polypeptide sequence of SEQ ID NO:152, and a light chain variable region having the polypeptide sequence of SEQ ID NO: 154; or
k. a heavy chain variable region having the polypeptide sequence of SEQ ID NO:153, and a light chain variable region having the polypeptide sequence of SEQ ID NO:154.

Embodiment 10 is an isolated monoclonal antibody or antigen-binding fragment thereof of any one of embodiments 1 to 9, wherein the monoclonal antibody or antigen-binding fragment thereof is capable of binding GITR and inducing effector-mediated tumor cell lysis.

Embodiment 11 is an isolated nucleic acid encoding the monoclonal antibody or antigen-binding fragment thereof of any one of embodiments 1 to 10.

Embodiment 12 is a vector comprising the isolated nucleic acid of embodiment 11.

Embodiment 13 is a host cell comprising the vector of embodiment 12.

Embodiment 14 is a pharmaceutical composition, comprising the isolated monoclonal antibody or antigen-binding fragment thereof of any one of embodiments 1 to 10 and a pharmaceutically acceptable carrier.

Embodiment 15 is a method of treating cancer in a subject in need thereof, comprising administering to the subject the pharmaceutical composition of embodiment 14.

Embodiment 16 is a method of targeting GITR on a cancer cell surface in a subject in need thereof, the method comprising administering to the subject the pharmaceutical composition of embodiment 14.

Embodiment 17 is a method of producing the monoclonal antibody or antigen-binding fragment thereof of any one of embodiments 1 to 10, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or culture.

Embodiment 18 is a method of producing a pharmaceutical composition comprising the monoclonal antibody or antigen-binding fragment of any one of embodiments 1 to 10, comprising combining the monoclonal antibody or antigen-binding fragment thereof with a pharmaceutically acceptable carrier to obtain the pharmaceutical composition.

Embodiment 19 is a method of determining a level of GITR in a subject, the method comprising:
a. obtaining a sample from the subject;
b. contacting the sample with an isolated monoclonal antibody or antigen-binding fragment thereof of any one of embodiments 1 to 10; and
c. determining a level of GITR in the subject.

Embodiment 20 is the method of embodiment 19, wherein the sample is a tissue sample or blood sample.

Embodiment 21 is the method of embodiment 20, wherein the tissue sample is a cancer tissue sample.

### EXAMPLES

The examples below are intended to be purely exemplary of the invention and should therefore not be considered to limit the invention in any way. The following examples and detailed description are offered by way of illustration and not by way of limitation.

### Example 1: Generation of Anti-GITR Monoclonal Antibodies (mAbs)

Immunization

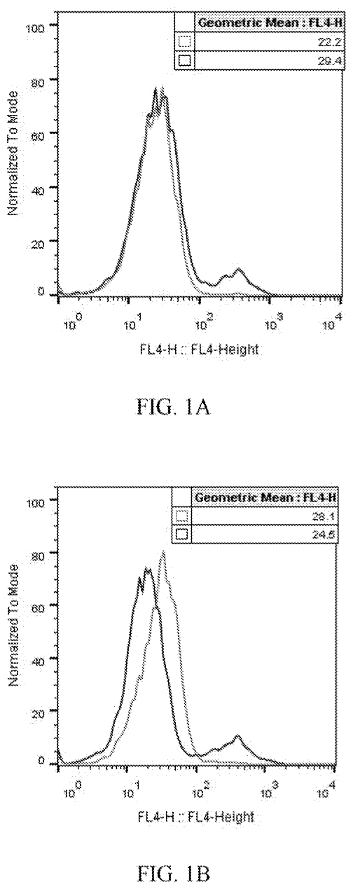

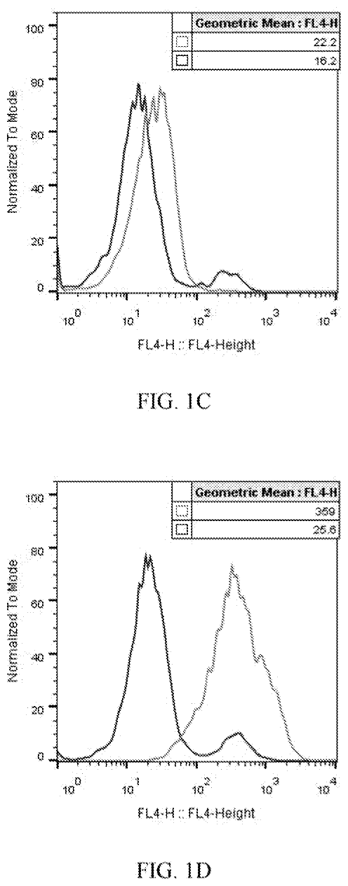

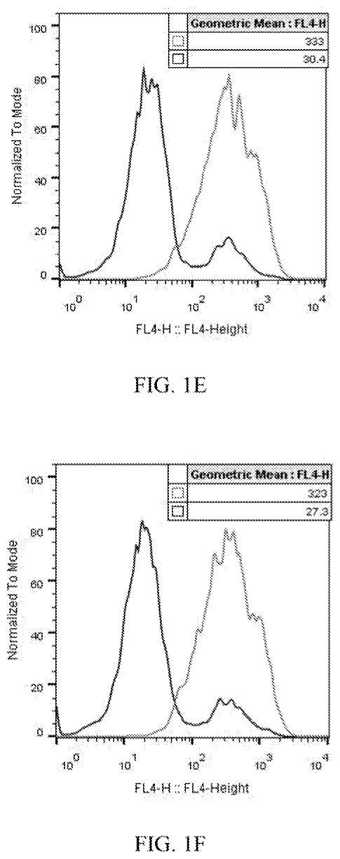

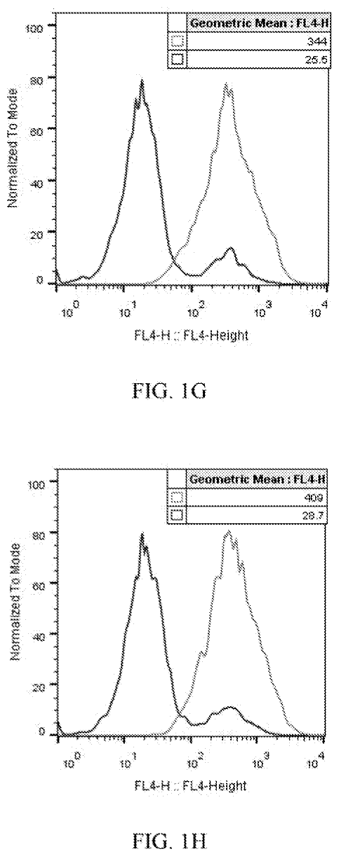

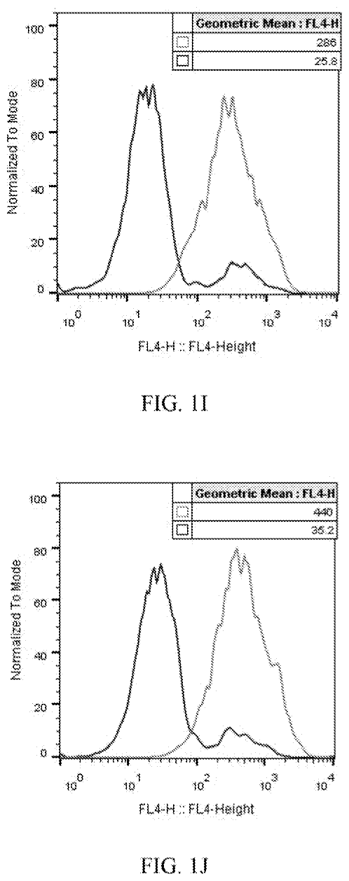

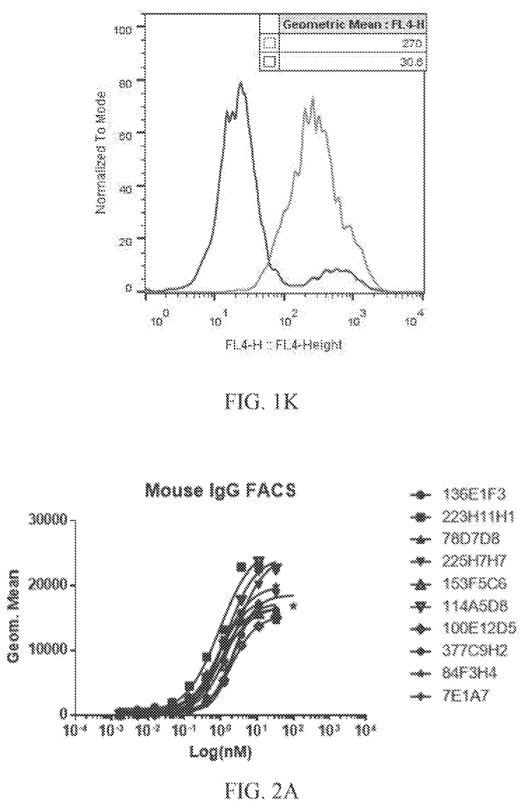

Balb/c and C57 mice were immunized with human GITR-Fc protein (GenScript; Cat #Z03440) under current animal welfare regulations. For immunization, the antigen was administrated in PBS solution or formulated as an emulsion with CFA (Complete Freund's adjuvant; primary immunization) or IFA (incomplete Freund's adjuvant; boost immunizations). In sets of immunization methods, the antigen was administered with a gene gun in the abdominal skin of the mouse, by intraperitoneal injection or subcutaneously at dorsal. Each animal received 4 doses (first one at 50 μg/mL, followed by 25 μg/mL each for the next three). 7 days after each time point during the immunization schedule, 20 μL of blood samples (FIGS. 1A-1K) were collected from the animals to monitor the anti-sera titer in an ELISA-based assay with immobilized GITR-Fc protein as a control, until the fusion criteria was met. The specific recognition of GITR-Fc was verified with ELISA of the binding to a biotin-GITR ligand-Fc.

Selections of Anti-GITR Antibody Secreting Hybridoma

Three days after the last immunization, splenocytes from the selected mouse were extracted and fused with sp2/0 cells following standard hybridoma generation protocol in a sterile environment. The fused cells were cultured in IX HAT (hypoxanthine-aminopterin-thymidine) containing DMEM media, supplemented with 10% FBS for 6 days. The contents in the supernatant were analyzed for binding ability to human GITR-Fc by ELISA with counter screening against human IgG1. Single positive clones against human GITR-Fc only were selected with a cut off value of OD₄₅₀>1. The selected clones were analyzed for their ability to bind GITR overexpressing Cho-K1 stable cell line by FACS. The positive parental clones were subcloned by limited dilution and cultured in 1×HT (hypoxanthine-thymidine) containing DMEM media, supplemented with 10% FBS. A 100 μL cell suspension was aliquoted to maintain 1-3 cells/well. Cells were cultured for 1 week before a new round of screening using ELISA and FACS, till positive monoclones were achieved. Each unique clone was selected to produce 0.5 mg of purified antibodies for further characterization. Antibody isotypes were tested (Clonotyping System-HRP, SouthernBiotech; Birmingham, Ala.) and antibodies were purified with Protein-A magnetic beads (GenScript, Cat #L00695), eluted by 0.5M Sodium-citrate solution (pH3.5), and neutralized with 0.5M Tris-HCl (pH9.0). The storage buffer was changed to PBS to determine concentration with nanodrops.

### Example 2: Characterization of Anti-GITR Mouse Antibodies in Vitro

GITR⁺/Cho-K1 Cell Binding by FACS

A total of 38 clones were sent for small scale production, and mAb products were generated, including 204C10012, 384H3H11, 289H17D4, 197E9G3, 45F1F3, 31D7D10, 136E1F3, 225H7H7, 100E12D5, 7E1A7, 263H6G11, 358E3C9, 222H1101, 206H9G11, 225A8D9, 223F12C4, 225H7012, 11A4E11, 223H11H1, 153F5C6, 377C9H2, 320E12H10, 209E10F11, 201A3A11, 274C7H2, 287F1E11, 275A4D7, 172H7B9, 163H12G7, 270C5C10, 78D7D8, 114A5D8, 114A5D8, 84F3H4, 366B6H9, 85D2H9, and 366G5C11.

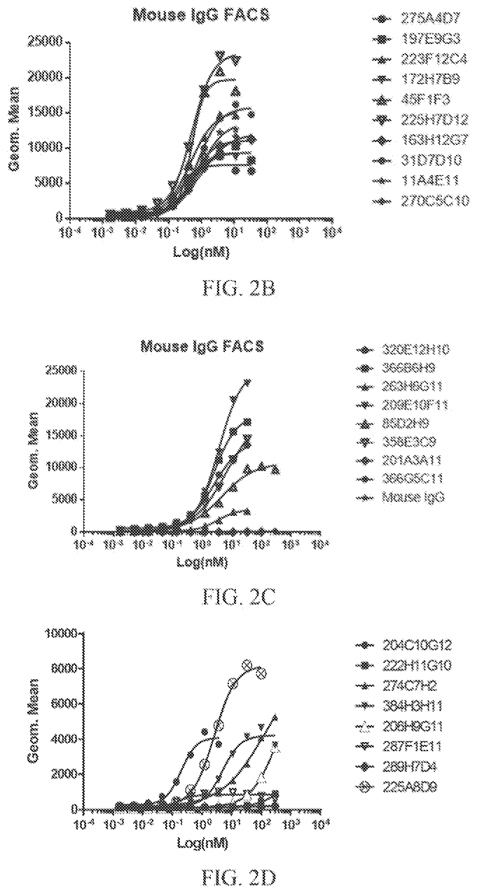

To verify cell surface antigen binding of antibody products, approximately 1×10⁵ cells/reaction of Cho-K1 cells expressing human GITR were harvested and incubated with a 3-fold serially diluted anti-GITR mAb from 10⁻³ to 10² mM, followed by the detection with 1 μg/mL of fluorophore (iFluor 647)-labeled goat anti mouse IgG (H+L) secondary antibodies. The results are presented in FIGS. 2A-2D. EC₅₀ values are summarized in Table 1. A total of 34 mAbs demonstrated an EC₅₀ less than 100 ng/ml, out of which 30 mAbs demonstrated an ECs less than 10 ng/ml.

TABLE 1
FACS binding data of anti-GITR mouse monoclonal antibodies

| | EC₅₀ | | EC₅₀ | | EC₅₀ |
|---|---|---|---|---|---|
| Clone ID | (ng/ml) | Clone ID | (ng/ml) | Clone ID | (ng/ml) |
| 204C10G12 | 0.199 | 222H11G10 | ~1879 | 274C7H2 | 140.5 |
| 384H3H11 | 4.967 | 206H9G11 | 374.9 | 287F1E11 | 0.172 |
| 289H7D4 | ~16199 | 225A8D9 | 2.602 | 275A4D7 | 0.2158 |
| 197E9G3 | 0.4378 | 223F12C4 | 0.4545 | 172H7B9 | 0.4622 |
| 45F1F3 | 0.5075 | 225H7D12 | 0.5217 | 163H12G7 | 0.5499 |
| 31D7D10 | 0.7703 | 11A4E11 | 0.9091 | 270C5C10 | 0.9121 |
| 136E1F3 | 0.9186 | 223H11H1 | 0.9311 | 78D7D8 | 0.9983 |
| 225H7H7 | 1.011 | 153F5C6 | 1.027 | 114A5D8 | 1.422 |
| 100E12D5 | 1.975 | 377C9H2 | 2.132 | 84F3H4 | 2.219 |
| 7E1A7 | 2.336 | 320E12H10 | 2.486 | 366B6H9 | 2.586 |
| 263H6G11 | 3.224 | 209E10F11 | 3.578 | 85D2H9 | 4.506 |
| 358E3C9 | 6.945 | 201A3A11 | ~0.03865 | 366G5C11 | ~94.41 |

Anti-GITR Mouse Antibody Functional Reporter Gene Assay

The anti-GITR mouse antibody functional screening assay was conducted using the reporter gene-based anti-GITR agonist assay kit developed by Promega Corp (Cat #CS184009; Promega; Madison, Wis.). The kit contains one effector cell line expressing GITR, as well as a luciferase reporter gene under the regulation of a promoter. Under the agonist effect of the anti-GITR antibody, the effector cells will be stimulated to elicit a pro-inflammatory response, which activates certain transcriptional factors that bind to the response element upstream of the luciferase reporter gene, allowing for the production of luciferase. Addition of the substrate will generate a luminescence signal, which can be detected.

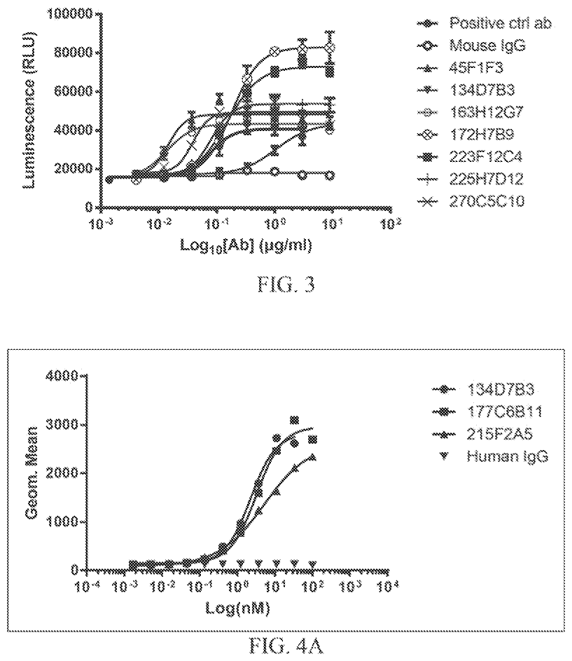

Briefly, the effector cell line expressing GITR was defrosted and counted, and its viability was tested. The cells were transferred to a 96 well plate at 50,000 cells per well. Antibody samples, anti-GITR human antibody (GenScript, B50011812), as a positive control, or mouse IgG, as a negative control, were prepared in a series of dilutions and added to the effector cells respectively and incubated for 6 hours at 37° C. with 5% CO₂, after which luciferase substrate solution was added to the mixture and the whole plate was incubated in the dark at room temperature for 10 minutes. Finally, the plate was read under Luminescence mode in PheraStar (BMGLabtech, PheraStarPlus FSX). If the antibody sample had an anti-GITR agonist effect, the luminescence signal showed an increasing sigmoidal curve against the antibody concentration (FIG. 3).

The EC₅₀ values are shown in Table 2. 134D7B3 did not show a good activity given the high EC₅₀. 172H719, 45F1F3, and 163H2G7 demonstrated a desirable EC₅₀ and are reasonable span.

TABLE 2
GITR functional reporter gene assay for
anti-GITR mouse monoclonal antibodies

| | EC₅₀ | | EC₅₀ | | EC₅₀ |
|---|---|---|---|---|---|
| Clone ID | (μg/ml) | Clone ID | (μg/ml) | Clone ID | (μg/ml) |
| 45F1F3 | 0.01446 | 134D7B3 | 1.05 | 163H12G7 | 0.01371 |
| 172H7B9 | 0.1815 | 223F12C4 | 0.1506 | 225H7D12 | 0.07961 |
| 270C5C10 | 0.03858 | Positive | 0.07691 | | |
| | | control | | | |

### Example 3: Antibody CDR Sequences Alignment

A total of 14 murine antibodies were sequenced. The CDR3 alignment results are summarized in Table 3, the CDR sequences am summarized in Table 4, and the sequences of the variable regions are shown below. The sequencing of three clones showed more than one heavy or light chain.

TABLE 3
Alignment of individual antibodies' CDR3 sequences

| Clone ID | CDRH3 | Clone ID | CDRL3 |
|---|---|---|---|
| 134D7B3 | FYYFGSSYAMDY | 134D7B3 | QQGNTLPFT |
| 177C6B11 | GGYYDYDGVAWLAP | 177C6B11 | QQYYSYPWT |
| 215F2A5 | REAMDY | 215F2A5 | FQGSHVPYT |
| 223H11H1 | REALDY | 223H11H1 | LQGSHIPWT |
| 204C10G12 | GDDFWYFDV | 204C10G12 | FQGSHVPLT |
| 206H9G11-Ha | RDYYAMDY | 206H9G11-La | QQHYSFPLT |
| 206H9G11-Hb | IAPITTVVATNYFDV | 206H9G11-Lb | QQSNSWLT |
| 225A8D9-H | SAWLGKTYVMDY | 225A8D9-L | QQWNFPFT |
| 274C7H2-H | NPSYDYYAMDY | 274C7H2-L | ALWYSNHFI |
| 45FIF3-H | SGHYDLFDY | 45FIF3-L | WQGTHFPYT |
| 163H12G7-H | WSYWYFDV | 163H12G7-L | YQATHFPLT |
| 172H7B9-H | RGSYSDYGGWSFDV | 172H7B9-La | QNDHSYPPT |
| 172H7B9-H | RGSYSDYGGWSFDV | 172H7B9-Lb | QNDYSYPLT |
| 172H7B9-H | RGSYSDYGGWSFDV | 172H7B9-Lc | QNDHSYPPT |
| 223F12C4-H | YIMDY | 223F12C4-L | HQGQDYPLT |
| 225H7D12-H | KASYYTMDY | 225H7D12-L | HQYHRSPRT |
| 270C5C10-Ha | PGSYGFAY | 270C5C10-L | WQGTHFPWT |
| 270C5C10-Hb | KDYDWFAY | 270C5C10-L | WQGTHFPWT |

TABLE 4
CDR sequences of mAb clones

| Clone, CDR | Amino Acid Sequence | SEQ ID NO: |
|---|---|---|
| 45F1F3 HCDR1 | IDYAWN | 36 |
| 45F1F3 HCDR2 | YISNSGYTSYNPSLKS | 37 |
| 45F1F3 HCDR3 | SGHYDLFDY | 38 |
| 45F1F3 LCDR1 | KSSQSLFDRDGKTYLS | 39 |
| 45F1F3 LCDR2 | LVSNLDS | 40 |
| 45F1F3 LCDR3 | WQGTHFPYT | 41 |
| 225H7D12 HCDR1 | GYWIE | 42 |
| 225H7D12 HCDR2 | EILPGSGVSNNNEKFRD | 43 |
| 225H7D12 HCDR3 | KASYYTMDY | 44 |
| 225H7D12 LCDR1 | TASSSVSSFYFH | 45 |
| 225H7D12 LCDR2 | SISNLAS | 46 |
| 225H7D12 LCDR3 | HQYHRSPRT | 47 |
| 270C5C10 HaCDR1 | DNYMN | 48 |
| 270C5C10 HaCDR2 | DINPNNGGSRYKEKFKD | 49 |
| 270C5C10 HaCDR3 | PGSYGFAY | 56 |
| 270C5C10 HbCDR1 | DYYMN | 51 |
| 270C5C10 HbCDR2 | IINPNNGDTSYNQKFKG | 52 |
| 270C5C10 HbCDR3 | RDYDWFAY | 53 |
| 270C5C10 LCDR1 | KSSQSLLDSDGRTYLN | 34 |
| 270C5C10 LCDR2 | LVSQLDS | 55 |
| 270C5C10 LCDR3 | WQGTHFPWT | 56 |
| 134D7B3 HCDR1 | NYVIE | 57 |
| 134D7B3 HCDR2 | VIYPGNGGSDYNEKFKV | 58 |
| 134D7B3 HCDR3 | FYYFGSSYAMDY | 59 |
| 134D7B3 LCDR1 | RASQDISNYLN | 60 |
| 134D7B3 LCDR2 | YTSRLHS | 61 |
| 134D7B3 LCDR3 | QQGNTLPFT | 62 |
| 177C6B11 HCDR1 | SFWMII | 63 |
| 177C6B11 HCDR2 | RIDPHTGHTKYNEKFKT | 64 |
| 177C6B11 HCDR3 | GGYYDYDGVAWLAF | 65 |
| 177C6B11 LCDR1 | KSSQSLLYSNQKNYLA | 66 |
| 177C6B11 LCDR2 | WASTRES | 67 |
| 177C6B11 LCDR3 | QQYYSYPWT | 68 |
| 215F2A5 HCDR1 | DHYMT | 69 |
| 215F2A5 HCDR2 | DINPYSGYTVIFNQRFKD | 76 |
| 215F2A5 HCDR3 | REAMDY | 71 |
| 215F2A5 LCDR1 | RSDQTIVYSNGNTYLQ | 72 |
| 215F2A5 LCDR2 | KVSNRFS | 73 |
| 215F245 LCDR3 | FQGSHVPYT | 74 |
| 223H11H1 HCDR1 | AHYMN | 75 |
| 223H11H1 HCDR2 | DINPDNGIIRYNQKFKD | 76 |
| 223H11H1 HCDR3 | REALDY | 77 |
| 223H11H1 LCDR1 | RSSQSIVYSNGKVYLN | 78 |
| 223H11H1 LCDR2 | KVSDRFS | 79 |
| 223H11H1 LCDR3 | LQGSHIPWT | 80 |
| 204C10G12 HCDR1 | SYWIT | 81 |
| 204C10G12 HCDR2 | DIYPGSDSTNYNEKFKN | 82 |
| 204C10G12 HCDR3 | GDDFWYFDV | 83 |
| 204C10G12 LCDR1 | RSSQSIVHSNGNTYLE | 84 |
| 204C10G12 LCDR2 | KVSNRFS | 85 |
| 204C10G12 LCDR3 | FQGSHVPLT | 86 |
| 206H9G11 HaCDR1 | DYYMH | 87 |
| 206H9G11 HaCDR2 | EINPSTGGTSYNQKFKA | 88 |
| 206H9G11 HaCDR3 | RDYYAMDY | 89 |
| 206H9G11 HbCDR1 | TFGMGVG | 90 |
| 206H9G11 HbCDR2 | HIWWDDDKYYNPALKS | 91 |
| 206H9G11 HbCDR3 | IAPITTVVATNYFDV | 92 |
| 206H9G11 LaCDR1 | KASQDVRIAVA | 93 |
| 206H9G11 LaCDR2 | SASYRFT | 94 |
| 206H9G11 LaCDR3 | QQHYSFPLT | 95 |
| 206H9G11 LbCDR1 | RASQSIGTSIH | 96 |
| 206H9G11 LbCDR2 | YASESIS | 97 |
| 206H9G11 LbCDR3 | QQSNSWLT | 98 |
| 225A8D9 HCDR1 | SSWMN | 99 |
| 225A8D9 HCDR2 | RIYPGDGNINYDGKFKG | 100 |
| 225A8D9 HCDR3 | SAWLGKTYVMDY | 101 |
| 225A8D9 LCDR1 | SASSSVTYMN | 102 |
| 225A8D9 LCDR2 | EISKLAS | 103 |
| 225A8D9 LCDR3 | QQWNFPFT | 104 |
| 274C7H2 HCDR1 | SYWMH | 105 |
| 274C7H2 HCDR2 | YINPSSGYTKYNQKFKD | 106 |
| 274C7H2 HCDR3 | NPSYDYYAMDY | 107 |
| 274C7H2 LCDR1 | RSSTGAVTTSNYAN | 108 |
| 274C7H2 LCDR2 | GTNNRAP | 109 |
| 274C7H2 LCDR3 | ALWYSNHFI | 110 |
| 383H3H11 HCDR1 | SYGVH | 111 |
| 383H3H11 HCDR2 | VIWSGGSTDYNAAFIS | 112 |
| 383H3H11 HCDR3 | IGLRSFAY | 113 |
| 383H3H11 LCDR1 | KSSQSLLNSGNQKNYLT | 114 |
| 383H3H11 LCDR2 | WASTRES | 115 |
| 383H3H11 LCDR3 | QNDYSYPFT | 116 |
| 163H12G7 HCDR1 | TYTVH | 117 |
| 163H12G7 HCDR2 | YINPASGYTNYNQKFKD | 118 |
| 163H12G7 HCDR3 | WSYWYFDV | 119 |
| 163H12G7 LCDR1 | KSSQSLLDSDGKTYLN | 120 |
| 163H12G7 LCDR2 | LVSKLDS | 121 |
| 163H12G7 LCDR3 | YQATHFPLT | 122 |
| 172H7B9 HCDR1 | PSGMGVT | 123 |
| 172H7B9 HCDR2 | HIYWDDVKRYNPSLKS | 124 |
| 172H7B9 HCDR3 | RGSYSDYGGWSFDV | 125 |
| 172H7B9 LaCDR1 | KSSQSLFNSGNQKNYLA | 126 |
| 172H7B9 LaCDR2 | GASTRES | 127 |
| 172H7B9 LaCDR3 | QNDHSYPPT | 128 |
| 172H7B9 LbCDR1 | KSSQSLLNSGNQKNYLT | 129 |
| 172H7B9 LbCDR2 | WASTRES | 130 |
| 172H7B9 LbCDR3 | QNDYSYPLT | 131 |
| 172H7B9 LcCDR1 | KSSQSLLNSGNQKNYLT | 132 |
| 172H7B9 LcCDR2 | WASTRES | 133 |
| 172H7B9 LcCDR3 | QNDHSYPPT | 134 |
| 223F12C4 HCDR1 | SYWIH | 135 |
| 223F12C4 HCDR2 | RIHPSDGDIDHNEKFKG | 136 |
| 223F12C4 HCDR3 | YIMDY | 137 |
| 223F12C4 LCDR1 | HASQNINVWLS | 138 |
| 223F12C4 LCDR2 | KAAKLQT | 139 |
| 223F12C4 LCDR3 | HQGQDYPLT | 140 |

### Example 4: Chimeric Antibody Production and Analysis

Construction of Chimeric Antibodies for Characterization

All of the candidates tested in FIG. 3, together with 204C10G12, 206H9G11, 225A8D9, 274C7H2, 384H3H11, 177C6811, and 215F2A5 were selected for chimeric antibody synthesis onto the human IgG1 constant regions and sequencing. Variable region coding sequences for these 14 mAbs were optimized for human codon biased expression with GenScript online tools. The variable DNA fragments of 45F1F3, 163H12G7, 172H7B9, 223F12C4, 225H7D12, 270C5C10, 204C10G12, 225A8D9, 274C7H2, 206H9G11, 134D7B3, 177C6B11, 215F2A5 and 384H3H11 were synthesized and fused to human IgG1 heavy chain domains (CH1-hinge-CH2-CH3) and light chain kappa constant regions (CL) for transient expression in chimeric formats. The variable DNA fragment of 45F1F3, 225H7D12, 270C5C10, 163H12G7, 172H7B9, and 223F12C4 were fused onto the human IgG1 constant regions with the modifications of K214R, D356E, L358M and ΔK447. The variable DNA fragment of 204C10G12, 225A8D9, 274C7H2, 384H3H11, 134D7B3, 177C6B11, 215F2A5, and 223H11H1 were fused onto human IgG1 constant regions with the modification of K214R. The heavy chain and light chain expression constructs were cloned into individual pTT5 based plasmids, which contain a synthesized signal peptide for secretory expression. Clone 206H9G11 failed to express, which was showing non clonality from sequencing.

The chimeric antibodies were expressed in HEK293-6E cells (National Research Council) transfected with antibody heavy chain/light pair plasmids using PEImax 40,000 (Polysciences, Inc.; Warrington, Pa.). After 24 hours, the expression/secretion was boosted with a Tryptone N-1 supplement. After 5 days of shaking culture at 37° C. and 5% CO₂, supernatants were collected, and the antibody content was purified with Protein-A beads described above. Chimeric antibody products were kept in PBS for analysis.

Chimeric Antibody FACS Binding Analysis

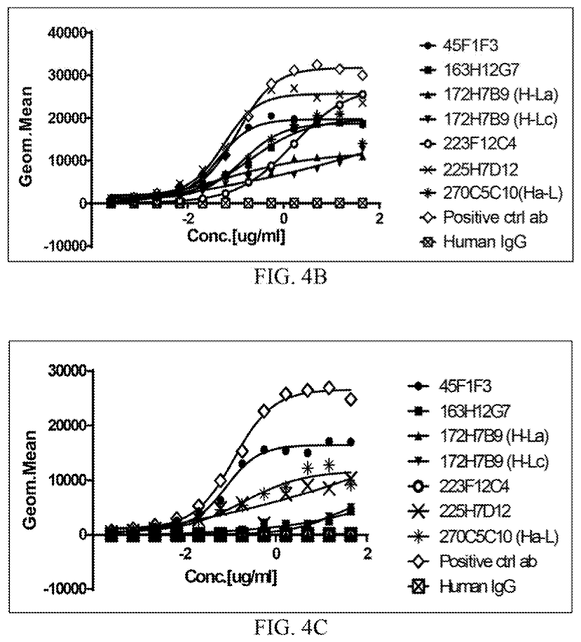

The binding pattern of chimeric antibodies with GITR expressed on Cho-K1 cells were plotted with antibody in 3× serial dilutions from a starting concentration of 300 nM. Antibody-GITR binding curves were generated with geometric mean values. Raw data was plotted with GraphPad Prism v6.02 software with four parameters, and a best-fit values program was used to analyze the EC₅₀ (FIGS. 4A-4C). Results are summarized in Table 5.

Cross species reactivity was tested for all candidates on FACS with a cynomolgus monkey GITR/Cho-K1 cell line (cyno GITR/Cho-K1). Non and weak cynomolgus binders were eliminated for further testing. 45F1F3, 225H7D12, and 270C5C10 were selected as lead candidates with desirable EC₅₀ from both human and cyno GITR/Cho-K1 FACS analysis. 45F1F3 and 225H7D12 both showed superior EC₅₀ compare to the benchmark antibody used as a positive control. 270C5C10 had slightly higher EC, and in the epitope binning experiment, 270C5C10 showed binding to a different epitope than 45F1F3 and 225H7D12. Thus, all three clones were chosen for further development.

TABLE 5
Binding of chimeric antibodies on GITR/Cho-K1 cells in FACS analysis

| | EC₅₀ | | EC₅₀ | | EC₅₀ |
|---|---|---|---|---|---|
| Clone ID | (μg/ml) | Clone ID | (μg/ml) | Clone ID | (μg/ml) |
| 45F1F3 | 0.04429 | 384H3H11 | 218.8 | 274C7H2 | ~330.8 |
| 225A8D9 | 0.2516 | 204C10G12 | ~19449 | 225H7D12 | 0.05656 |
| 172H7B9(H-La) | 0.04994 | 172H7B9(H-Lc) | ~3.340e+024 | 223F12C4 | 1.592 |
| 163H12G7 | 0.2099 | 270C5C10(Ha-L) | 0.1384 | Positive | 0.1121 |
| | | | | control | |

Chimeric Ani-GITR Antibody Functional Reporter Gene Assay

The EC₅₀ values of three candidate chimeric antibodies from a GITR functional reporter gene assay, as described in Example 2, are summarized in Table 6. Clone 45F1F3 shows the lowest EC₅₀ value of 0.01446 μg/ml.

TABLE 6
Chimeric antibody GITR functional reporter gene assay

| | 45F1F3 | 225H7D12 | 270C5C10(Ha-L) | Positive Ctrl Ab |
|---|---|---|---|---|
| Bottom | 16310 | 16819 | 17967 | 15802 |
| Top | 48289 | 53784 | 49466 | 40806 |
| LogEC₅₀ | −1.84 | −1.099 | −1.414 | −1.114 |
| Hill Slope | 2.879 | 2.402 | 3.066 | 2.215 |
| EC₅₀ (μg/ml) | 0.01446 | 0.07961 | 0.3858 | 0.07691 |
| Span | 31979 | 36974 | 31499 | 25004 |

### Example 5. Humanization Antibody Production and Analysis

Humanization Design for the Candidate Antibodies

Based on antibody variable domain sequences, the CDRs, HV loops and FRs were analyzed and homology modeling was performed to obtain the modeled structure of the mouse antibody. The solvent accessible surface area of framework residues was calculated. Based on the result, identify framework residues that are buried (i.e. with solvent accessible surface area of <15%) were identified. One human acceptor for VH and VL that shared the top sequences identical to the mouse counterparts was selected. The CDRs of the mouse antibody were directly grafted to the human acceptor frameworks to obtain the sequence of the grafted antibody without any back mutation, where certain amino acids were changed back to murine framework sequences. For candidate 45F1F3, 4 VH sequences and 2 VL sequences were synthesized with back mutations. For candidate 225H7D12, 4 VH sequences and 3 VL sequences were synthesized with back mutations. For candidate 270C5C10, 5 VH sequences and 2 VL sequences were synthesized with back mutations. Post translational modifications and chemical degradation were analyzed in the grafted sequences, including deamidation, isomerization oxidation, and glycosylation through a developability assessment. PTM hotspots like N-glycosylation sites, unusual proline residues, deamidation sites, isomerization sites, oxidation sites, and unpaired cysteine residues were identified that could affect the binding activity and manufacturability of the grafted antibody.

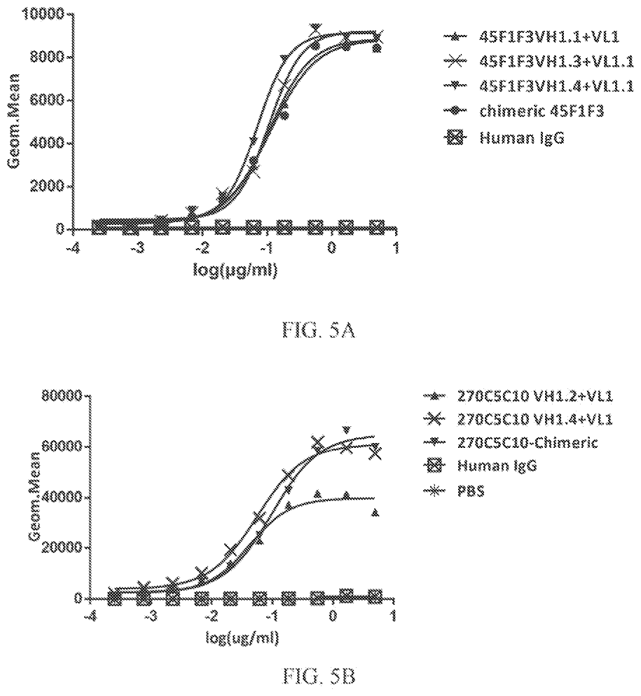

The DNA sequences encoding the humanized light and heavy chain variable regions were synthesized. The antibody characteristics were compared to select the best candidate. For each chimeric antibody, one humanized antibody was chosen as the final lead based on a low EC₅₀ from a FACS binding assay with GITR/Cho-K1 and the least number of back mutations (FIGS. 5A-5C). EC₅₀ values are summarized in Table 7. All three humanized antibodies for chimeric antibody 45F1F3 demonstrated a lower EC₅₀. Both humanized antibodies for chimeric antibody 270C5C10 demonstrated a lower EC₅₀. Two humanized antibodies for chimeric antibody 225H7D12 demonstrated a lower EC₅₀, except VH1.4+VL1.2 where the EC₅₀ was 0.1810 μg/mL compared to 0.1122 μg/mL before humanization.

TABLE 7
FACS binding EC₅₀ of humanized antibodies.

| | EC₅₀ | | EC₅₀ | | EC₅₀ |
|---|---|---|---|---|---|
| Clone ID | (μg/ml) | Clone ID | (μg/ml) | Clone ID | (μg/ml) |
| 45F1F3 | 0.1072 | 270C5C10 | 0.04243 | 225H7D12 | 0.07548 |
| VH1.1 + VL1 | | VH1.2 + VL1 | | VH1.1 + VL1.2 | |
| 45F1F3 | 0.1059 | 270C5C10 | 0.05584 | 225H7D12 | 0.08198 |
| VH1.3 + VL1.1 | | VH1.4 + VL1 | | VH1.3 + VL1.2 | |
| 45F1F3VH1.4 + | 0.07044 | 270C5C10 | 0.09822 | 225H7D12 | 0.1810 |
| VL1.1 | | chimeric | | VH1.4 + VL1.2 | |
| 43F1F3 chimeric | 0.1114 | | | 225H7D12 chimeric | 0.1122 |

Cell Based Reporter Gene Assay for the Humanized Antibodies

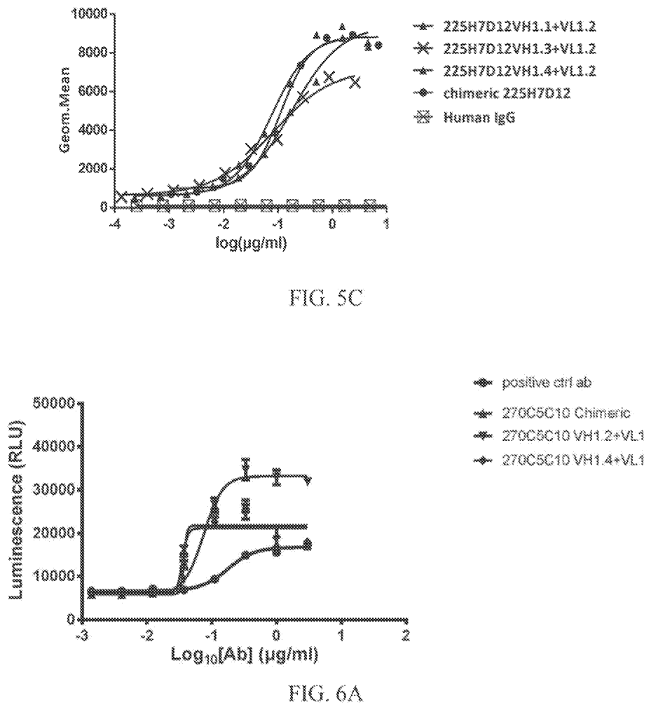

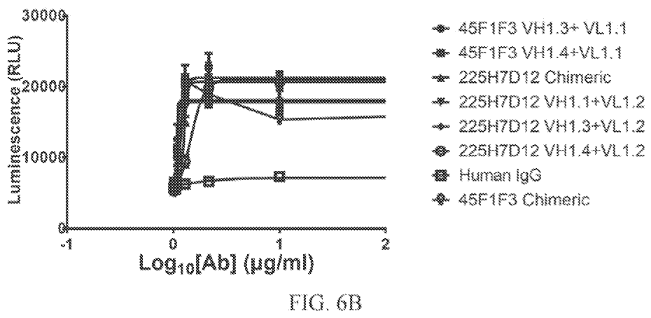

The anti-GITR antibody functional screening was conducted using the reporter gene-based anti-GITR agonist assay developed by GenScript. The assay includes one effector cell line expressing GITR, as well as a luciferase reporter gene under the regulation of a promoter. Under the agonist effect of an anti-GITR antibody, the effector cells were stimulated to elicit a pro-inflammatory response, which activated NFkB transcription factors that bind to the response element upstream of the luciferase reporter gene, allowing for the production of luciferase. Addition of the substrate generated a luminescence signal, which be recorded by a luminometer. The functional bioassay result for all humanized lead candidates, their corresponding chimeric antibodies and positive control anti-GITR antibody (Genscript, Anti-GITRAb BA20190125CFT02) are shown in FIGS. 6A and B. All 11 candidate antibodies tested showed greater potency than the benchmark antibody. Candidate 270C5C10 VL1+VH1.4 showed the lowest EC₅₀ (0.03552 μg/mL), which was greater than 5 times more potent than the benchmark antibody with an EC₅₀ of 0.1781 μg/mL (Table 8).

TABLE 8
Humanized antibodies GITR functional reporter gene assay

| | EC₅₀ | | EC₅₀ | | EC₅₀ |
|---|---|---|---|---|---|
| Clone ID | (μg/ml) | Clone ID | (μg/ml) | Clone ID | (μg/ml) |
| 45F1F3 | 1.085 | 270C5C10 | 0.07449 | 225H7D12 | 1.172 |
| VH1.3 + VL1.1 | | VH1.2 + VL1 | | VH1.1 + VL1.2 | |
| 45F1F3 | 1.093 | 270C5C10 | ~0.03552 | 225H7D12 | 1.184 |
| VH1.4 + VL1.1 | | VH1.4 + VL1 | | VM1.3 + VL1.2 | |
| 45F1F3 chimeric | ~1.093 | 270C5C10 | ~0.03736 | 225H7D12 | 1.457 |
| | | chimeric | | VH1.4 + VL1.2 | |
| Positive control | 0.1781 | Human IgG | 0.9966 | 225H7D12 chimeric | 1.264 |
| Ab | | | | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Sequence Listing
mAb clone 45F1F3-H Heavy chain variable region
SEQ ID NO: 1
DVQLQESGPGLVKPSQSLSLTCTVTGYSITIDYAWNWIRQFPGNKLEWMGYISNGSYTSYNPSLKS
RISFTRDTSKNQFFLQLNSVTPEDSATYYCTRSGHYDLFDYWGQGTTLTVSS
mAb clone 45F1F3-L Light chain variable region
SEQ ID NO: 2
DVVMTQAPLTLSVTIGQPASISCKSSQSLFDRDGKTYLSWLLERPGQSPKRLIYLVSNLDSGVPDRF
TGSGSGTDFTLKISRVEAEDLGVYYCWQGTHFPYTFGGGTKVEMK
mAb clone 225H7D12-H Heavy chain variable region
SEQ ID NO: 3
QVQLQQSGAEVMKPGSVKLSCKTTGYRFTGYWIEWVKQRPGHGLEWIGEILPGSGVSNNNEKF
RDKATFTADTSSNTAYIQLSSLTTDDSAIYYCARKASYYTMDYWGQGTSVTVSS
mAb clone 225H7D12-L Light chain variable region
SEQ ID NO: 4
QIVLTQSPAIMSASLGERVTMTCTASSSVSSFYFHWYQQKPGSSPKLWIYSISNLASGVPTRFSGSGS
GTSYSLTISSMEAEDAATYYCHQYHRSPRTFGGGTKLEIK
mAb clone 270C5C10-Ha Heavy chain variable region a (VHa)
SEQ ID NO: 5
EVQLQQSGPEVVKPGASVKISCKASGYTFTDNYMNWAKQSQGKSLEWIGDINPNNGGSRYKEKF
KDKATLTVDKSSRTAYMELRSLTRSEDSAVYYCANPGSYGFAYWGQGRLVTVSA
mAb clone 270C5C10-Hb Heavy chain variable region b (VHb)
SEQ ID NO: 6
EVQLQQSGPVQVKPGASVKMSCKASGFTFDYYMNWVKQSHGKSLEWIGIINPNNGDTDYNQKF
KGKATLTVDKSSSTAYMELNSLTSEDSAVYYCARRDYDWFAYWGQGTLVTVSA
mAb clone 270C5C10-L Light chain variable region (VL)
SEQ ID NO: 7
DVVLTQTPLTLSVTLGQPASISCKSSQSLLDSDGRTYLNWLLQRPGQSPKRLIYLVSQLDSGVPDRF
TGSGSGTDFTLKISRVEAEDLGVYFCWQGTHFPWTPGGGTKLEIK
mAb clone 134D7B3-H Heavy chain variable region (VH)
SEQ ID NO: 8
QVQLQQSGAELVRPGTSVKVSCKASGYAFNNYVIEWVKQRPGQGLEWIGVIYPGNGGSDYNEKF
KVKAKLTADKSSSTAYMQLSSLTSDDSAVYFCARFYYFGSSYAMDYWGQGTSVTVSS
mAb clone 134D7B3-L Light chain variable region (VL)
SEQ ID NO: 9
DIQMTQTTSSLSASLGDRVTISCRASQDISNYLNLNWYQQKPDGTIKLLIYYTSRLHSGVPSRFSGSGS
GTDYSLTITNLEQEDIAATYFCQQGNTLPFTFGSGTKLEIK
mAb clone 177C6B11-H Heavy chain variable region (VH)
SEQ ID NO: 10
QVQLQQPGAELVKPGASVKLSCKASGYFTSFWMHWVKQRPGRGLEWIGRIDPHTGGTKYNEKF
KTKATLTVDKPSSAAYMQLSSLTSEDSAVYYCARGGYYDYDGVAWLAFWGQGTLVTVSA
mAb clone 177C6B11-L Light chain variable region (VL)
SEQ ID NO: 11
DIVMSQSPSSLAVSVGEKVTMSCKSSQSLLYSSNQKNYLAWYQQKPGQSPKLLIYWASTRESGVP
DRFTGSGSGTDFTLTISSVKAEDLAVYYCQQYYSYPWTFGGGTKLEIK
mAb clone 215F2A5-H Heavy chain variable region (VH)
SEQ ID NO: 12
EVQLQQSGPDLVKPGTSVKISCKASGYTLSDHYMTWVKQSHGKSLEWIADINPYSGTVTFNQRFK
DKATLTVDTSSSTAFMELRSLTSEDSAVYYCARREAMDYWGQGTSVTVSS
mAb clone 215F2A5-L Light chain variable region (VL)
SEQ ID NO: 13
GVLMTQTPLSLPVSLGDQASISCRSDQTIVYSNGNTYLQWYLQRPGQSPRLLIYKVSNRFSGVPDR
FSGSGSGTDFTLKISRVEAEDLGVYYCFQGSHVPYRFGGGTRLEIK
mAb clone 223H11H1-H Heavy chain variable region (VH)
SEQ ID NO: 14
EVQLQQSGPELVKPGASVKISCKASGYTFTAHYMNWVKKSQGKSLEWIGDINPDNGIIRYNQKFK
DKATLTVDKSSSTVFLEFRSLASEDSAVYYCARREALDYWGQGSSVTVSS
mAb clone 223H11H1-L Light chain variable region (VL)
SEQ ID NO: 15
DVLMTQIPLSLTVSLGDQASTSCRSSQSIVYSNGKVYLNWYQQKPGQSPKLLIYKVSDRFSGVPDRF
SGSGSGTDFTLKINRVEAEDLGVYYCLQGSHIPWTFGGGTKLEIK
mAb clone 204C10G12-H Heavy chain variable region (VH)
SEQ ID NO: 16
QVQLQQPGAELKPGASVKMSCKASGYTFTSYWITWVKQRPGQGLEWIGDIYPGSDSTNYNEKF
KNKATLTVDTSSSTAYMQLSSLTSEDSAVYYCARGDDFWYFDVWGTGTTVTVSS
mAb clone 204C10G12-L Light chain variable region (VL)
SEQ ID NO: 17
DVLMTQTPLSLPVSLGDQASISICRSSQSIVHSNGNTYLEWYLQKPGQSPKLLIYKVSNRFSGVPDRF
SGSGSGTDFTLKISRVEAEDLGVYYCFQGSHVPLTFGAGTKLELK
mAb clone 206H9G11-Ha Heavy chain variable region 1 (VH1)
SEQ ID NO: 18
EVQKQQSGPALKPGASVKISCKASGYSFTDYYMHMKQSPEKSLEWIGEINPSTGGTSYNQKFK
AKATLTVDKSSNTAYMQLKSLTSEDSAVYFCTRRDYYAMDYWDQGTSVIVSS
mAb clone 206H9G11-Hb Heavy chain variable region 2 (VH2)
SEQ ID NO: 19
QVTLKESGPGILQPSQTLSLTCSFSGFSLSTFGMGVGWIRQPSGKGLEWLAHIWWDDDKYYNPAL
KSRLTISKDTSKNQVFLKIANVDTADTATYYCARIAPITTVVATNYFDVWGTGTTVTVSS
mAb clone 206H9G11-La Light chain variable region 1 (VL1)
SEQ ID NO: 20
DIVMTQSHKFMSTSVGDRVNITCKASQDVRIAVAWYQQKPGQSPKLLIYSASYRFTGVPDRFTGS
GSGTDFTFTISSVQAEDLAVYYCQQHYSFPLTFGAGTKLELT
mAb clone 206H9G11-Lb Light chain variable region 2 (VL2)
SEQ ID NO: 21
DILLTQSPAILSVSPGERVSFSCRASQSIGTSIHWYQQRTNGSPRLLIKYASESISGIPSRFSGSGSGTD
FTLSINSVESEDIADYYCQQSNSWLTFGAGTKLELK
mAb clone 225A8D9-H Heavy chain variable region (VH)
SEQ ID NO: 22
QVQLQQSGPELVKPGASVKISCKASGYAFSSSWMNWVKQRPGKGLEWIGRIYPGDGNINYDGKF
KGEATLTADKSSSTAYIQLSSLTSEDSAVYFCARSAWLGKTYVMDYWGQGTSVTVSS
mAb clone 225A8D9-L Light chain variable region (VL)
SEQ ID NO: 23
EIVVTQSPAITAASLGQKVTITCSASSSVTYMNWYQQKSGTSPKPWIYEISKLASGVPAHFSGSGSG
TSYSLTISSLEAEDAAIYYCQQWNFPFTFGSGTKVEIR
mAb clone 274C7H2-H Heavy chain variable region (VH)
SEQ ID NO: 24
QVQKQQSGAELAKPGASVKLSCKASGYTFTSYWMHWVKQRPGQGLEWIGYINPSSGQTKYNQKF
KDKATLTADKSSSTAYMQLSSLTYEDSAVYYCARNPSYDYYAMDYWGQGTSVTVSS
mAb clone 274C7H2-L Light chain variable region (VL)
SEQ ID NO: 25
QAVVTQESALTTSPGETVTLTCRSTGAVTTSNYANWVQEKPDHLFTGLIGGTNNRAPGVPARFS
GSLIGDKAALTTTGAQTEDEAIYFCALWYSNHFIFGSGTKVTVL
mAb clone 384H3H11-H Heavy chain variable region (VH)
SEQ ID NO: 26
QVQLKQSGPGLVQPSQSLSTTCTVSGFSLTSYGVHWVRQSPGKGLEWLGVIWSGGSTDYNAAFISR
LSISKDNSKSQVFFKMNSLQANDTAIYYCARIGLRSFAYWGQGTLVTVSA
mAb clone 384H3H11-L Light chain variable region (VL)
SEQ ID NO: 27
DIVMTQSPSSLTVTGEKVTMSCKSSQSLLNSGNQKNYLTWYQQKPGQPPKLLIYWASTRESGVP
DRFTGSGSGTDFTLTISSVQAEDLAVYYCQNDYSYPFTFGSGTKLEIK
mAb clone 163H12G7-H Heavy chain variable region (VH)
SEQ ID NO: 28
QVQLQQSGAELASPGASVKMSCKASGYTFSTYTVHWVKQRPGQGLEWIGYINPASGYTNYNQKF
KDRATLTADRSSTTAYMQLSGLTSEDSAVFYCARWSYWYFDVWGTGTTVTVSS
mAb clone 163H12G7-L Light chain variable region (VL)
SEQ ID NO: 29
DVVMTQTPLTLSVSIGQPASISCKSSQSLLDSDGKTYLNWFLQRPGQSPKRLLYLVSKLDSGVPDR
FTGSGSGTDFTLKISRVEAEDLGVYYCYQATHFPLTPGAGTKLELK
mAb clone 172H7B9-H Heavy chain variable region (VH)
SEQ ID NO: 30
QVTLKESGPGILQSSQTLSLTCSFSGFSLSPSGMGVTWIRQPSGKGLEWLAHIYWDDVKRYNPSLK
SRLTISKDTSRNQVFLKITGVDTADTATYYCARRGSYSDYGGWSFDVWGTGTTVTVSS
mAb clone 172H7B9-La Light chain variable region a (VLa)
SEQ ID NO: 31
DIVMTQSPSSLSVSAGEKVTMSCKSSQSLFNSGNQKNYLAWYQQKPGQPPKLLIYGASTRESGVP
DRFTGSGSGSDFTLTISSVQAEDLAVYYCQNDHSYPPTFGSGTKLEIK
mAb clone 172H7B9-Lb Light chain variable region b (VLb)
SEQ ID NO: 32
DIVMTQSPSSLTVTAGEKTMSCKSSQSLLNSGNQKNYLTWYQQKPGQPPKLLIYWASTRESGVP
DRFTGSGSGSTDFTLTISSVQAEDLAVYYCQNDYSYPLTFGAGTKLELK
mAb clone 172H7B9-Lc Light chain variable region c (VLc)
SEQ ID NO: 33
DIVMTQSPSSLTVTAGEKVTMSCKSSQSLLNSGNQKNYLTWYQQKPGQPPKLLIYWASTRESGVP
DRFTGSGSGTDFTLTISSVQAEDLAVYYCQNDHSYPPTFGSGTKLEIK
mAb clone 223F12C4-H Heavy chain variable region (VH)
SEQ ID NO: 34
QVQLQQSGAELVKPGASVKVSCKASGYSFTSYWIHWVKQRPGQGLEWIGRIHPSDGDIDHNEKFK
GKATLTVDKSSSTAYMQLTSLTSEDSAVYYCLCYIMDYWGQGTSVTVSS
mAb clone 223F12C4-L Light chain variable region (VL)
SEQ ID NO: 35
DIQMNQSPSSLSASLGDTIAITCHASQNINVWLSWYQQKPGNIPKLLIYKAAKLQTGVPSRFSGSGS
GTGFTLTISSLQPEDIATYYCHQGQDYPLTFGAGTKLELK
Human IgG1 CH
SEQ ID NO: 141
ASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPPELLGGPSVFLFPPKPKDTL
MISRTPEVTCVVVDVSHEDPEVKFNWYVDGVEVHNAKTKPREEQYNSTYRVVSVLTVLHQDWL
NGKEYKCKVSKALPAPIEKTISKAKGQPREPQVYTLPPSRDELTKNQVSLTCLVKGFYPSDIAVE
WESNGQPENNYKTTPPVLDSGSFFLYSKLTVDKSRWQQGNVFSCSVMHEALHNHYTQKSLSLSP
GK
Human IgG1 CH variant 1
SEQ ID NO: 155
ASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSP
G
Human IgG1 CH variant 2
SEQ ID NO: 156
ASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTKSLSLSP
GK
Human IgG1 CL
SEQ ID NO: 142
RTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
humanized mAb 45F1F3 VH1.1
SEQ ID NO: 143
QVQLQESGPGLVKPSQTLSLTCTVTGYSITIDYAWNWIRQPPGKGLEWIGYISNSGYTSYNPSLKSR
VTFSDTSKNQFSLKLSSVTAADTAVYYCTRSGHYDLFDYWGQGTTVTVSS
humanized mAb 45F1F3 VH1.3
SEQ ID NO: 144
QVQLQESGPGLVKPSQTILSLTCTVTGYSITIDYAWNWIRQPPGKGLEWMGYISNSGYTSYNPSLKS
RVTISRDTSKNQFFLKLSSVTAADTAVYYCTRSGHYDLFDYWGQGTTVTVSS
humanized mAb 45F1F3 VH1.4
SEQ ID NO: 145
DVQLQESGPGLVKPSQTLSLTCTVTGYSITIDYAWNWIRQPPGKGLEWMGYISNSGYTSYNPSLKS
RITFSRDTSKNQFFLKLSSVTAADTAVYYCTRSGHYDLFDYWGQGTTVTVSS
humanized mAb 45F1F3 VL1
SEQ ID NO: 146
DVVMTQSPLSLPVTLGQPASISCKSSQSLFDRDGKTYLSWFQQRPGQSPRRLIYLVSNLDSGVPDRF
SGSGSGTDFTLKISRVEAEDVGVYYCWQGTHFPYTFPYTFGGGTKVEIK
humanized mAb 45F1F3 VL1.1
SEQ ID NO: 147
DVVMTQSPLSLPVTLGQPASISCKSSQSLFDRDGKTYLSWLLQRPGQSPRRLIYLVSNLDSGVPDRF
SGSGSGTDFTLKISRVEAEDVGVYYCWQGTHFPYTFGGGTKVEIK
humanized mAb 225H7D12 VH1.1
SEQ ID NO: 148
QVQLVQSGAEVKKPGASVKVSCKTTGYRFTGYWIEWVRQAPGQGLEWIGEILPGSGVSNNNEKF
RDRATMTADTSISTAYMELSRLRSDDTAVYYCARKASYYTMDYWGQGTLVTVSS
humanized mAb 225H7D12 VH1.3
SEQ ID NO: 149
QVQLVWSGAEVKKPGASVKVSCKASGYRFTGYWIEWVKQAPGQGLEWIGEILPGSGVSNNNEKF
RDRATMTADTSINTAYMELSRLRSDDTAVYYCARKASYYTMDYWGQGTLVTVSS
humanized mAb 225H7D12 VH1.4
SEQ ID NO: 150
QVQLVQSGAEVKKPGASVKVSCKTGYRFTGYWIEWVKQAPGQGLEWIGEILPGSGVSNNNEKF
RDRATFTADTSINTAYMELSRLRSDDTAVYYCARKASYYTMDYWGQGTLVTVSS
humanized mAb 225H7D12 VH1.2
SEQ ID NO: 151
DIQMTQSPSSLSASVGDRVTITCTASSSVSSFYFHWYQQKPGKAPKLWIYSISNLASGVFSGSG
SGTSYTLTISSLQPEDFATYYCHQYHRSPRTFGGGTKLEIK
humanized mAb 270C5C10 VH1.2
SEQ ID NO: 152
QVQLVQSGAEVKKPGASVKVSCKASGYTFTDNYMNWVRQAPGQSLEWIGDINPNNGGSRYKEK
FKDRATLTVDTSIRTAYMELSRLRSDDTAVYYCANPGSYGFAYWGQGTLVTVSS
humanized mAb 270C5C10 VH1.4
SEQ ID NO: 153
QVQKVQSGAEVKKPGASVKVSCKASGYTFTDNYMNWARQSPGQSLEWIGDINPNNGGSRYKEKF
KDRATLTVDTSIRTAYMELSRLRSDDTAVYYCANPGSYGFAYWGQGTLVTVSS
humanized mAb 270C5C10 VL1
SEQ ID NO: 154
DVVMTQSPLSLPVTLGQPASISCKSSQSLLDSDGRTYLNWFQQRPGQSPRRLIYLVSQLDSGVPDRF
SGSGSGTDFTLKISRVEAEDVGYYCWQGTHFPWTFGGGTKLEIK

1. An isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequence of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
or a variant thereof comprising up to about three amino acid substitutions in HCDR1, HCDR2, HCDR3, LCDR1, LCDR2, and/or LCDR3;
wherein the antibody or antigen-binding fragment thereof specifically binds glucocorticoid-induced tumor necrosis factor receptor related protein (GITR), preferably human GITR.

2. The isolated monoclonal antibody or antigen-binding fragment thereof of claim 1, comprising a heavy chain complementarity determining region 1 (HCDR1), HCDR2, HCDR3, a light chain complementarity determining region 1 (LCDR1), LCDR2, and LCDR3, having the polypeptide sequence of:
a. SEQ ID NOs: 36, 37, 38, 39, 40, and 41, respectively;
b. SEQ ID NOs: 42, 43, 44, 45, 46, and 47, respectively;
c. SEQ ID NOs: 48, 49, 50, 54, 55, and 56, respectively;
d. SEQ ID NOs: 51, 52, 53, 54, 55, and 56, respectively;
e. SEQ ID NOs: 57, 58, 59, 60, 61, and 62, respectively;
f. SEQ ID NOs: 63, 64, 65, 66, 67, and 68, respectively;
g. SEQ ID NOs: 69, 70, 71, 72, 73, and 74, respectively;
h. SEQ ID NOs: 75, 76, 77, 78, 79, and 80, respectively;
i. SEQ ID NOs: 81, 82, 83, 84, 85, and 86, respectively;
j. SEQ ID NOs: 87, 88, 89, 93, 94, and 95, respectively;
k. SEQ ID NOs: 87, 88, 89, 96, 97, and 98, respectively;
l. SEQ ID NOs: 90, 91, 92, 93, 94, and 95, respectively;
m. SEQ ID NOs: 90, 91, 92, 96, 97, and 98, respectively;
n. SEQ ID NOs: 99, 100, 101, 102, 103, and 104, respectively;
o. SEQ ID NOs: 105, 106, 107, 108, 109, and 110, respectively;
p. SEQ ID NOs: 111, 112, 113, 114, 115, and 116, respectively;
q. SEQ ID NOs: 117, 118, 119, 120, 121, and 122, respectively;
r. SEQ ID NOs: 123, 124, 125, 126, 127, and 128, respectively;
s. SEQ ID NOs: 123, 124, 125, 129, 130, and 131, respectively;
t. SEQ ID NOs: 123, 124, 125, 132, 133, and 134, respectively; or
u. SEQ ID NOs: 135, 136, 137, 138, 139, and 140, respectively;
wherein the antibody or antigen-binding fragment thereof specifically binds glucocorticoid-induced tumor necrosis factor receptor related protein (GITR), preferably human GITR.

3. The isolated monoclonal antibody or antigen-binding fragment thereof of claim 1, comprising a heavy chain variable region having a polypeptide sequence at least 95%, at least 96%, at least 97%, at least 98%, or at least 99% identical to SEQ ID NO: 1, 3, 5, 6, 8, 10, 12, 14, 16, 22, 24, 26, 28, 30, or 34, or a light chain variable region having a polypeptide sequence at least 95%, at least 96%, at least 97%, at least 98%, or at least 99% identical to SEQ ID NO: 2, 4, 7, 9, 11, 13, 15, 17, 23, 25, 27, 29, 31, 32, 33, or 35.

4. The isolated monoclonal antibody or antigen-binding fragment thereof of claim 1, comprising:
a. a heavy chain variable region having the polypeptide sequence of SEQ ID NO:1, and a light chain variable region having the polypeptide sequence of SEQ ID NO:2;
b. a heavy chain variable region having the polypeptide sequence of SEQ ID NO:3, and a light chain variable region having the polypeptide sequence of SEQ ID NO:4;
c. a heavy chain variable region having the polypeptide sequence of SEQ ID NO:5, and a light chain variable region having the polypeptide sequence of SEQ ID NO:7;
d. a heavy chain variable region having the polypeptide sequence of SEQ ID NO:6, and a light chain variable region having the polypeptide sequence of SEQ ID NO:7;
e. a heavy chain variable region having the polypeptide sequence of SEQ ID NO:8, and a light chain variable region having the polypeptide sequence of SEQ ID NO:9;
f. a heavy chain variable region having the polypeptide sequence of SEQ ID NO:10, and a light chain variable region having the polypeptide sequence of SEQ ID NO:11;
g. a heavy chain variable region having the polypeptide sequence of SEQ ID NO:12, and a light chain variable region having the polypeptide sequence of SEQ ID NO:13;
h. a heavy chain variable region having the polypeptide sequence of SEQ ID NO:14, and a light chain variable region having the polypeptide sequence of SEQ ID NO:15;
i. a heavy chain variable region having the polypeptide sequence of SEQ ID NO:16, and a light chain variable region having the polypeptide sequence of SEQ ID NO:17;
j. a heavy chain variable region having the polypeptide sequence of SEQ ID NO:22, and a light chain variable region having the polypeptide sequence of SEQ ID NO:23;
k. a heavy chain variable region having the polypeptide sequence of SEQ ID NO:24, and a light chain variable region having the polypeptide sequence of SEQ ID NO:25;
l. a heavy chain variable region having the polypeptide sequence of SEQ ID NO:26, and a light chain variable region having the polypeptide sequence of SEQ ID NO:27;
m. a heavy chain variable region having the polypeptide sequence of SEQ ID NO:28, and a light chain variable region having the polypeptide sequence of SEQ ID NO:29;
n. a heavy chain variable region having the polypeptide sequence of SEQ ID NO:30, and a light chain variable region having the polypeptide sequence of SEQ ID NO:31;
o. a heavy chain variable region having the polypeptide sequence of SEQ ID NO:30, and a light chain variable region having the polypeptide sequence of SEQ ID NO:32;
p. a heavy chain variable region having the polypeptide sequence of SEQ ID NO:30, and a light chain variable region having the polypeptide sequence of SEQ ID NO:33; or
q. a heavy chain variable region having the polypeptide sequence of SEQ ID NO:34, and a light chain variable region having the polypeptide sequence of SEQ ID NO:35.

5. The isolated monoclonal antibody or antigen-binding fragment thereof of claim 1, wherein the antibody or antigen-binding fragment thereof is chimeric.

6. The isolated monoclonal antibody or antigen-binding fragment thereof of claim 5, wherein the monoclonal antibody or antigen-binding fragment thereof comprises the human IgG1 constant regions.

7. The isolated monoclonal antibody or antigen-binding fragment thereof of claim 6, wherein the human IgG1 constant regions comprise at least one amino acid modification(s) selected from K214R, D356E, L358M, and ΔK447.

8. The isolated monoclonal antibody or antigen-binding fragment thereof of claim 5, wherein the antibody or antigen-binding fragment thereof is human or humanized.

9. The isolated humanized monoclonal antibody or antigen-binding fragment thereof of claim 8, comprising:
a. a heavy chain variable region having the polypeptide sequence of SEQ ID NO:145, and a light chain variable region having the polypeptide sequence of SEQ ID NO:147;
b. a heavy chain variable region having the polypeptide sequence of SEQ ID NO:153, and a light chain variable region having the polypeptide sequence of SEQ ID NO:154
c. a heavy chain variable region having the polypeptide sequence of SEQ ID NO:148, and a light chain variable region having the polypeptide sequence of SEQ ID NO:151;
d. a heavy chain variable region having the polypeptide sequence of SEQ ID NO:143, and a light chain variable region having the polypeptide sequence of SEQ ID NO:146;
e. a heavy chain variable region having the polypeptide sequence of SEQ ID NO:143, and a light chain variable region having the polypeptide sequence of SEQ ID NO:147;
f. a heavy chain variable region having the polypeptide sequence of SEQ ID NO:144, and a light chain variable region having the polypeptide sequence of SEQ ID NO:146;
g. a heavy chain variable region having the polypeptide sequence of SEQ ID NO:144, and a light chain variable region having the polypeptide sequence of SEQ ID NO:147;
h. a heavy chain variable region having the polypeptide sequence of SEQ ID NO:145, and a light chain variable region having the polypeptide sequence of SEQ ID NO:146;
i. a heavy chain variable region having the polypeptide sequence of SEQ ID NO:149, and a light chain variable region having the polypeptide sequence of SEQ ID NO:151;
j. a heavy chain variable region having the polypeptide sequence of SEQ ID NO:150, and a light chain variable region having the polypeptide sequence of SEQ ID NO:151; or
k. a heavy chain variable region having the polypeptide sequence of SEQ ID NO:152, and a light chain variable region having the polypeptide sequence of SEQ ID NO:154.

10. The isolated monoclonal antibody or antigen-binding fragment thereof of claim 1, wherein the monoclonal antibody or antigen-binding fragment thereof is capable of binding GITR and inducing effector-mediated tumor cell lysis.

11. An isolated nucleic acid encoding the monoclonal antibody or antigen-binding fragment thereof of claim 1.

12. A vector comprising the isolated nucleic acid of claim 11.

13. A host cell comprising the vector of claim 12.

14. A pharmaceutical composition, comprising the isolated monoclonal antibody or antigen-binding fragment thereof of claim 1 and a pharmaceutically acceptable carrier.

15. A method of treating cancer in a subject in need thereof, comprising administering to the subject the pharmaceutical composition of claim 14.

16. A method of targeting GITR on a cancer cell surface in a subject in need thereof, the method comprising administering to the subject the pharmaceutical composition of claim 14.

17. (canceled)

18. A method of producing a pharmaceutical composition comprising the monoclonal antibody or antigen-binding fragment of claim 1, comprising combining the monoclonal antibody or antigen-binding fragment thereof with a pharmaceutically acceptable carrier to obtain the pharmaceutical composition.

19. A method of determining a level of GITR in a subject, the method comprising:
a. obtaining a sample from the subject;
b. contacting the sample with an isolated monoclonal antibody or antigen-binding fragment thereof of claim 1; and
c. determining a level of GITR in the subject.

20. The method of claim 19, wherein the sample is a tissue sample or blood sample.

21. The method of claim 20, wherein the tissue sample is a cancer tissue sample.
